(19) United States
(12) Patent Application Publication
(10) Pub. No.: US 2004/0236349 A1
(43) Pub. Date: Nov. 25, 2004

(54) Cervical tenaculum

(76) Inventors: Barry N. Gellman, N. Easton, MA (US); Armand Morin, Berkley, MA (US); Jozef Slanda, Milford, MA (US); Michael Barenboym, Framingham, MA (US)

Correspondence Address: Fay Kaplun & Marcin, LLP, Suite 702, 150 Broadway, New York, NY, 10038, US

(21) Appl. No.: 10/761,651

(22) Filed: Jan. 21, 2004

Related U.S. Application Data
(60) Provisional application No. 60/441,929, filed on Jan. 22, 2003.
(60) Provisional application No. 60/465,697, filed on Apr. 25, 2003.

(51) Int. Cl. A61B 17/42

(52) U.S. Cl. 606/119

(57) ABSTRACT

A method and device for sealing a patient's cervix around a medical device is described. The device comprises a tenaculum including a base with a device receiving opening extending therethrough and a plurality of arms extending between proximal ends connected to the base and distal ends adapted to apply radial pressure to the cervix in combination with an arm closing element slidable along the arms between open and closed positions. An alternative cervical sealing device comprises an elongated frame with a distal end for placement adjacent to a cervix. The elongated frame defines a device receiving passage and a constriction element coupled to a distal end thereof. The constriction element is operable between a constricted and open configurations for selectively applying a radially inwardly directed force to the cervix. A manual control actuates the constriction element between the constricted and open configurations.

### INCORPORATION BY REFERENCE

The entire disclosures of U.S. Provisional Application Ser. No. 60/441,929, filed Jan. 22, 2003, and U.S. Provisional Application Ser. No. 60/465,697, filed Apr. 25, 2003, including the specification, claims and abstract of each are hereby expressly incorporated by reference herein.

### BACKGROUND

Accessing the uterus to perform diagnostic and/or therapeutic procedures often requires dilation of the cervix to facilitate the introduction of instruments and to reduce trauma. However, fluid or gas leakage can occur when the cervix is over dilated or patulated, or during manipulation of a device accessing the uterus. For example, a number of gynecologic procedures involve accessing the uterus through the cervix and applying intracavity pressure and/or circulation of medium for treatment. Such procedures include, for example, uterine ablation using, for example, an RF uterine ablation system, or a heated saline ablation system such as the HydroTherm Ablator® (HTA®), in addition to procedures such as hystosalphingogram, hydroablation of the uterine lining and uterine dilation during hysteroscopic examination. The cervix muscle is and strong and often creates an effective seal. However, such procedures often require mechanically reinforcing the cervix closure to prevent fluid or gas leakage therefrom.

Limitations to the natural sealing of the cervix around medical tools inserted thereinto occurs from over dilatation, weak muscle tone, or from movement of the device within the cervical os. To assist in maintaining the applied pressure (e.g., from about 50 mmHg to about 80 mmHg or more) during therapeutic and/or diagnostic procedures, a clamp may be applied about the cervix to improve sealing and prevent bypass.

Current methods of compressing the cervix include tenaculums that clamp externally around the cervix, suture loops that lasso the cervix tightly around an instrument and purse string sutures woven in and out of the cervix and drawn tightly to restrict the cervix and apply compression about the instrument. Conventional tenaculums include scissors-like clamps that apply a significant amount of compression to the cervix. However, multiple clamps are often required to provide sufficient localized pressure points to seal the cervix around its entire circumference. Suture loops are often suitable only where there is substantial cervix protrusion enabling the loop to lasso and secure the cervix.

### SUMMARY OF THE INVENTION

In one aspect, the present invention is directed to a cervical tenaculum comprising a base including a device receiving opening extending therethrough an a plurality of arms, each arm extending from a proximal end connected to the base to a distal end adapted to apply radial pressure to a cervix in combination with an arm closing element slidable along the arms between an open position in which the distal ends of the arms are released to a radially expanded configuration and a closed position in which the distal ends of the arms are radially constricted by the arm closing element with respect to the open position.

The present invention is further directed to a cervical sealing device, comprising an elongated frame with a distal end for placement adjacent to a cervix, the elongated frame defining a device receiving passage extending therethrough in combination with a constriction element coupled to the distal end of the elongated frame, the constriction element being operable between a constricted configuration for applying a radially inwardly directed force to the cervix and an open configuration in which the constriction element is loosened around the cervix and a manual control actuating the constriction element between the constricted and open configurations.

### BRIEF DESCRIPTION OF DRAWINGS

FIG. 1 is a side elevation view of an embodiment of a cervical tenaculum according to the invention;

FIG. 2 is a perspective view of another embodiment of a cervical tenaculum according to the invention;

FIG. 3 is a different perspective view of the cervical tenaculum shown in FIG. 2;

FIG. 4 is a perspective view of an arm closing subassembly of a cervical tenaculum according to the invention;

FIG. 5 is a perspective view of a device lock according to the invention;

FIG. 6 is a perspective view of an inner ring of a cervical tenaculum according to the invention;

FIG. 7 is a perspective view of an arm closing ring of a cervical tenaculum according to the invention;

FIG. 8 is a perspective view of a lock subassembly of a cervical tenaculum according to the invention;

FIG. 9 is a perspective view of a cam lock of a cervical tenaculum according to the invention;

FIG. 10 is a perspective view of a cam lock base of a cervical tenaculum according to the invention;

FIG. 11 is a perspective view of a clamp arm ring of a cervical tenaculum according to the invention;

FIG. 12 is a perspective view of an arm of a cervical tenaculum according to the invention;

FIG. 13 is a diagram showing a cervical tenaculum according to the invention with an inserted medical device;

FIG. 14 is a diagram showing the cervical tenaculum of FIG. 13 in the open position;

FIG. 15 is a diagram showing a detail of the arms of the cervical tenaculum according to the invention;

FIG. 16 is a diagram showing a cervical tenaculum in the closed position with an inserted device according to the invention;

FIG. 17 is a perspective diagram showing a cervical tenaculum according to an embodiment of the invention;

FIG. 18 is a diagram showing a first spring of a cervical tenaculum according to the invention;

FIG. 19 is a diagram showing a cervical tenaculum in the closed position applied to a cervix according to the invention;

FIG. 20 is a diagram showing a cervical tenaculum in the closed position with an inserted device according to the invention;

FIG. 21 is a diagram showing another embodiment of a cervical tenaculum with a loop device according to the invention;

FIG. 22 is a diagram showing the cervical tenaculum of FIG. 21 in the open position applied to a cervix;

FIG. 23 is a diagram showing the cervical tenaculum of FIG. 21 in the closed position applied to a cervix;

FIG. 24 is a perspective diagram of another embodiment of a cervical tenaculum with a planar spring in the closed configuration, according to the invention;

FIG. 25 is a front view diagram of the cervical tenaculum of FIG. 24 in a closed position;

FIG. 26 is a diagram of the tenaculum shown in FIG. 24, disposed on a simulated cervix in a closed configuration;

FIG. 27 is a diagram of the tenaculum shown in FIG. 24, disposed on a simulated cervix in an open configuration;

FIG. 28 is a perspective diagram of the tenaculum shown in FIG. 24, in the open configuration;

FIG. 29 is a front view diagram of the tenaculum shown in FIG. 24, in the open configuration;

FIG. 30 is a front view diagram of another embodiment of a cervical tenaculum having a guide ring according to the invention;

FIG. 31 is a perspective view of the cervical tenaculum shown in FIG. 30;

FIG. 32 is a perspective diagram of the cervical tenaculum shown in FIG. 30 placed on a simulated cervix in the closed configuration;

FIG. 33 is a perspective diagram of the cervical tenaculum shown in FIG. 30 placed on a simulated cervix in the open configuration;

FIG. 34 is a front view diagram of the cervical tenaculum shown in FIG. 30 placed on a simulated cervix in the open configuration;

FIG. 35 is a perspective diagram of a different embodiment of a cervical tenaculum having a loop and two control arms according to the invention;

FIG. 36 is a side view diagram of the cervical tenaculum shown in FIG. 35 with an inserted medical device in the open configuration;

FIG. 37 is a side view diagram of the cervical tenaculum shown in FIG. 35 with an inserted medical device in the closed configuration;

FIG. 38 is a top view of a first embodiment of a cervical tenaculum comprising a linked clamp according to the present invention;

FIG. 39 is a top view of a second embodiment of a cervical tenaculum comprising a linked clamp according to the present invention;

FIG. 40 is a top view of a third embodiment of a cervical tenaculum comprising a linked clamp according to the present invention;

FIG. 41 is a side view of the clamp shown in FIG. 38;

FIG. 42 is a side view of the clamp shown in FIG. 39;

FIG. 43 is a side view of the clamp shown in FIG. 40;

FIG. 44 is a top view of the clamp shown in FIG. 40 connected to a forceps;

FIG. 45 is a top view of another embodiment of a cervical tenaculum comprising a tie with ratchets according to the invention;

FIG. 46 shows a diagram of the clamps shown in FIGS. 38-40 placed in the open position on a simulated cervix;

FIG. 47 shows a diagram of the clamps shown in FIGS. 38-40 placed in the closed position on a simulated cervix;

FIG. 48 shows a diagram of the clamps shown in FIGS. 38-40 placed in the closed and locked position on a simulated cervix;

FIG. 49 shows a more detailed diagram of the clamp of FIG. 38 placed on a simulated cervix;

FIG. 50 shows a more detailed diagram of the clamp of FIG. 39 placed on a simulated cervix;

FIG. 51 shows a more detailed diagram of the clamp of FIG. 40 placed on a simulated cervix;

FIG. 52 shows the clamp of FIG. 38 connected to a forceps;

FIG. 53 shows the clamp of FIG. 39 connected to a forceps;

FIG. 54 shows the clamp of FIG. 40 connected to a forceps;

FIG. 55 shows an additional embodiment of a cervical clamp having a ratchet according to the invention;

FIG. 56 shows the cervical clamp of FIG. 55 connected to a forceps;

FIG. 57 shows yet another embodiment of a cervical clamp including a double ratchet according to the invention;

FIG. 58 shows an additional embodiment of a cable tie cervical clamp according to the invention;

FIG. 59 is a perspective view of a different embodiment of a cinch-type cervical clamp according to the invention;

FIG. 60 shows side views of alternate distal ends of the clamp shown in FIG. 59 according to the invention;

FIG. 61 shows detailed view of the distal end of clamps shown in FIG. 60;

FIG. 62 shows a first embodiment of a press-to-seal clamp according to the present invention; and

FIG. 63 shows a second embodiment of a press-to-seal clamp according to the invention.

### DETAILED DESCRIPTION

The present invention may be further understood with reference to the following description and the appended drawings, wherein like elements are referred to with the same reference numerals. The present invention is related to medical devices used to access the uterus for medical treatment. In particular, the present invention relates to devices for retaining the seal formed by the cervix around a medical instrument introduced into the uterus. U.S. Pat. No. 5,980,534 to Gimpelson, which is incorporated herein by reference in its entirety, describes related procedures. As described above, this application also incorporates herein by reference the entirety of pending U.S. Application Ser. No. 60/441,929, filed Jan. 22, 2003 and U.S. Provisional Application Ser. No. 60/465,697, filed Apr. 25, 2003, both of which are assigned to SciMed Life Systems, Inc.

Exemplary embodiments of a cervical tenaculum according to the invention include multiple arms that simultaneously clamp around the cervix to provide a substantially uniform seal around the circumference thereof. The exemplary cervical tenaculum clamps to the cervix to prevent fluids or gases from leaking therefrom during procedures. The exemplary cervical tenaculum is also designed to attach to a device inserted within the uterus, such as a sheath or an endoscope, to resist relative movement of the tenaculum and the device. According to the invention, the tenaculum may be adapted to fit a variety of conventional scope and/or sheath models. Additionally, the invention may include a mechanism for quickly opening the arms of the cervical tenaculum, if rapid removal of the tenaculum or of the inserted device is necessary. The exemplary cervical tenaculum may also be adapted to fit axially over the inserted medical device, enabling good cervix visibility with minimal space constraints. Those of skill in the art will understand that clamping the cervix represents one exemplary use of the devices according to the invention, and that such devices may also be used in conjunction with other body structures into which an elongated medical device is to be inserted.

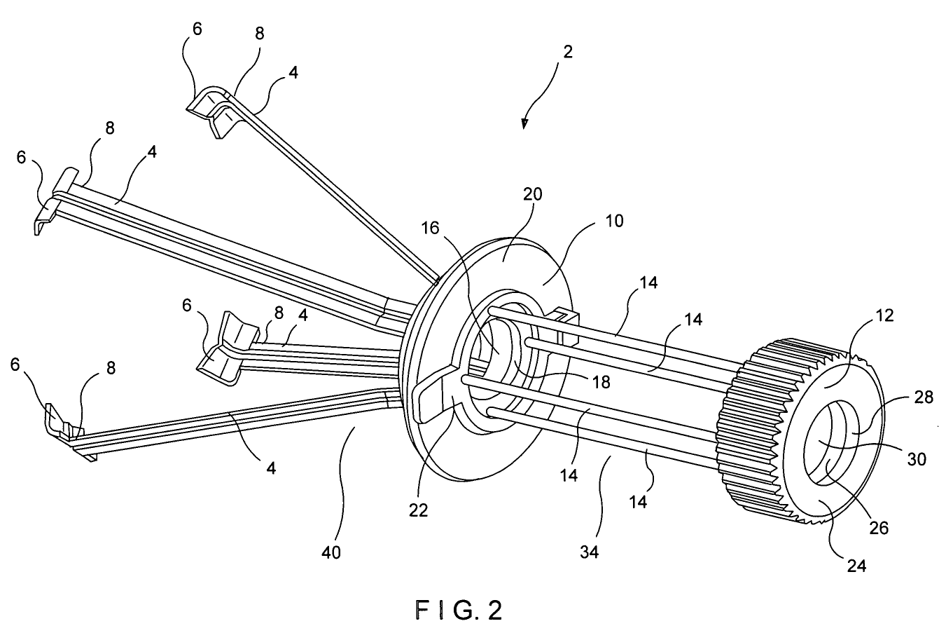

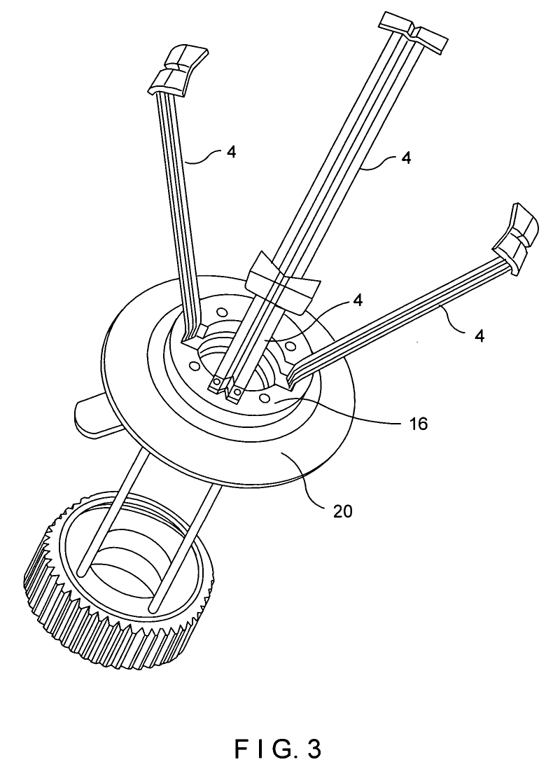

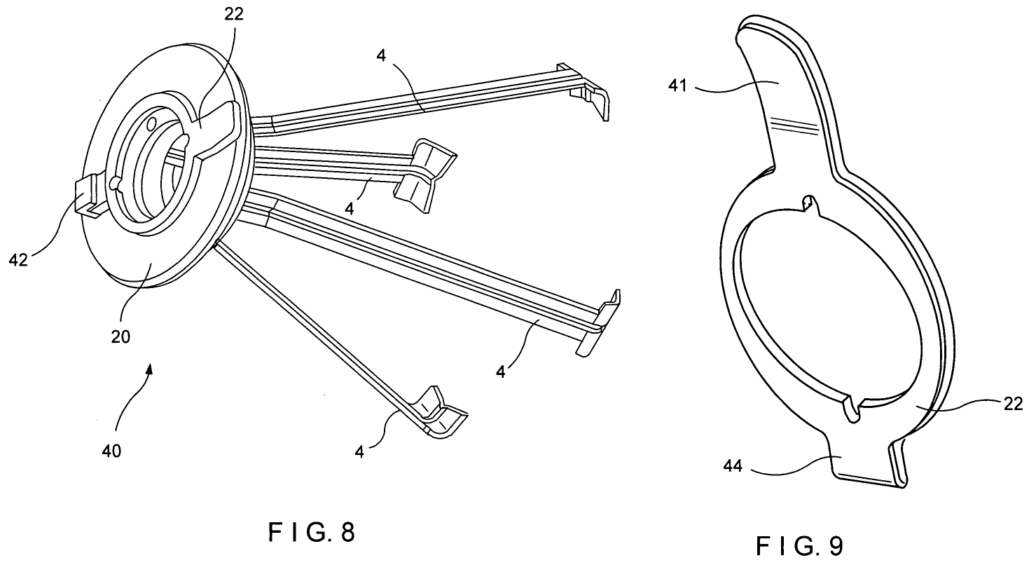

A first exemplary embodiment of a cervical tenaculum according the present invention is shown in FIGS. 2 and 3, which show the device in perspective from two different orientations. The cervical tenaculum 2 preferably includes multiple arms, e.g., four arms 4, each having an end-effector 6 on a distal end 8 thereof. In addition, the tenaculum 2 includes an arm lock 10, a device lock 12, and four guide rods 14. The arm lock 10 may further include an arm closing ring 16 which, when advanced toward the end-effectors 6, closes the arms 4 and, consequently, closes the end-effectors 6 around the cervix. The tenaculum 2 may also include a clamp arm ring 18 adjacent to the arm closing ring 16, and a cam lock base 20 disposed adjacent to the clamp arm ring 18. The cam lock base 20 is adapted to receive a cam lock 22 mounted adjacent thereto. Details of these elements are shown more clearly in FIG. 8.

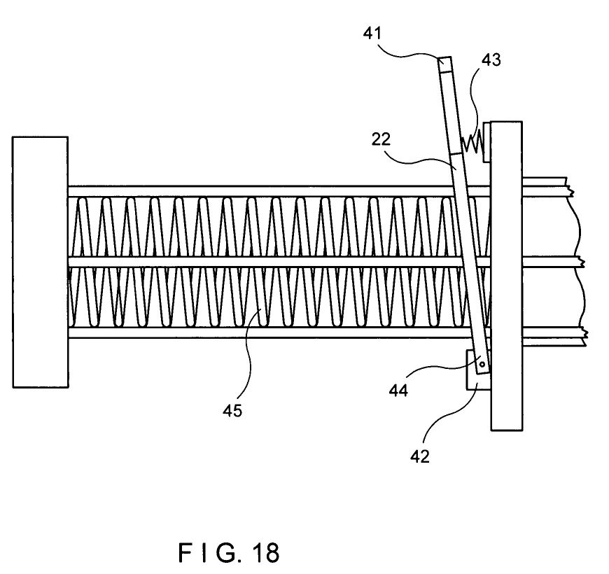

FIG. 18 shows additional details of the cam lock 22, which in one embodiment may be biased to a first position by a cam spring 43 disposed between the cam lock 22 and the cam lock base 20. The guide rods 14 extend through the cam lock 22 and the cam lock base 20 to reach the arm closing ring 16. In a first position, the cam spring 43 forces the cam lock 22 against at least one of the guide rods 14, such that the arm closing ring 16 cannot move. In a second position, the cam lock 22 is urged toward the cam lock base 20, compressing the cam spring 43 and releasing the cam lock 22 from the guide rod(s) 14.

In the embodiment depicted, the device lock 12 includes an outer ring 24 and an inner ring 26. The outer ring 24 and the inner ring 26 rotate around the same axis with an outer ring hole 28 offset from an inner ring hole 30 so that the outer ring 24 can be rotated relative to the inner ring 26 to reduce a size of an aperture between the holes 28 and 30. This feature may be used to effectively lock in place a medical device such as a sheath or a scope inserted through the device lock 12 by providing radial interference. The device lock 12, guide rods 14 and the arm closing ring 16 together define an arm closing subassembly 34 of the cervical tenaculum 2. Furthermore, each arm 4 of the cervical tenaculum 2 may preferably be formed as a leaf spring with each end-effector 6 being compressed around the cervix by advancing the arm closing ring 16 distally relative to the arms 4. Thus, multiple arms 4 are closed simultaneously with the movement of a single arm closing ring 16. However, those skilled in the art will understand that the length of each of the arms 4 may be substantially the same as that of the others so that, as the arm closing ring 16 is advanced distally, the end-effectors 6 will apply compression in a single plane. Alternatively, the lengths of the arms 4 may vary with respect to one another so that compression is applied substantially simultaneously at various sites along the cervix.

In another embodiment, the cervical tenaculum 2 may include a button mechanism which, when compressed, rapidly releases the arm lock 10. Alternatively, the release may be effectuated by other configurations that allow release of the arm lock 10 by a rapid and easy to execute movement of the user, such as by a single activation of an actuator. The cervical tenaculum 2 according to exemplary embodiments may include at least two arms 4, and can be operated by the user with only one hand. Furthermore, the cervical tenaculum 2 may be reusable or disposable, and, as would be understood by those of ordinary skill in the art, may be made of any suitable material such as, for example, steels or plastics using powder molding or liquid metal molding processes. The cervical tenaculum 2 according to embodiments of the invention may be used, for example, in conjunction with the Hydro ThermoAblator® (HTA®) uterine endometrial ablation system as well as a number of different procedures, including hystosaphingogram, hydroablation of the uterine lining, and uterine dilation during hysteroscopic examination.

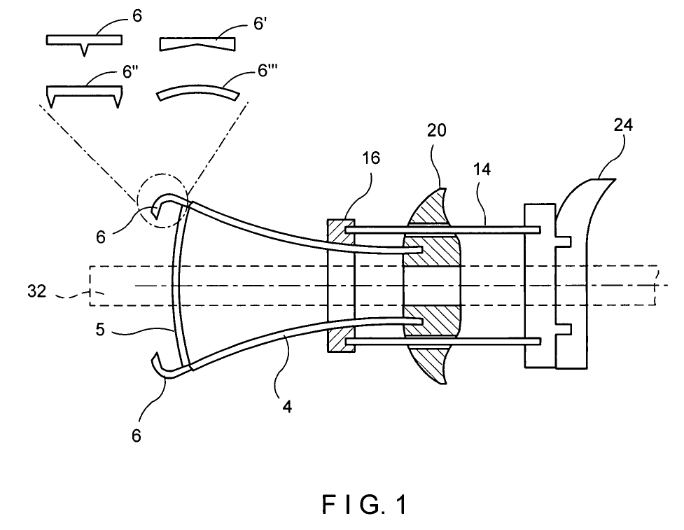

FIG. 1 shows an illustrative side elevation view of a cervical tenaculum 2 according to the present invention, including a detailed view of four end-effectors 6 according to exemplary embodiments of the invention. As shown, the end-effectors 6 may comprise pointed ends for grasping the cervix, or may be flat and long for increased force distribution across a larger area of contact with the cervical tissue. The end-effectors 6 may also comprise any combination of these shapes to achieve both the grasping modality and the force distribution modality desired. The end-effectors 6 may be removable (for example they may comprise a plastic sleeve which is slid over the ends of the arms 4) or they may be molded together with the distal ends 8 of the arms 4. The shape of the end-effectors 6 may be varied depending on the preference of each user, and the device and/or procedure being used. In an alternate exemplary embodiment which will be described below, the end-effectors 6 may include a snare-like configuration providing a range of approximately 350 degrees to greater than 360 degrees of loop to cinch about the cervix. This embodiment comprises a monofilarnent or braided stainless steel wire or suture coupled to an individual locking nut on the wire or suture. Wire 5 shown in FIG. 1 is an example of such a loop.

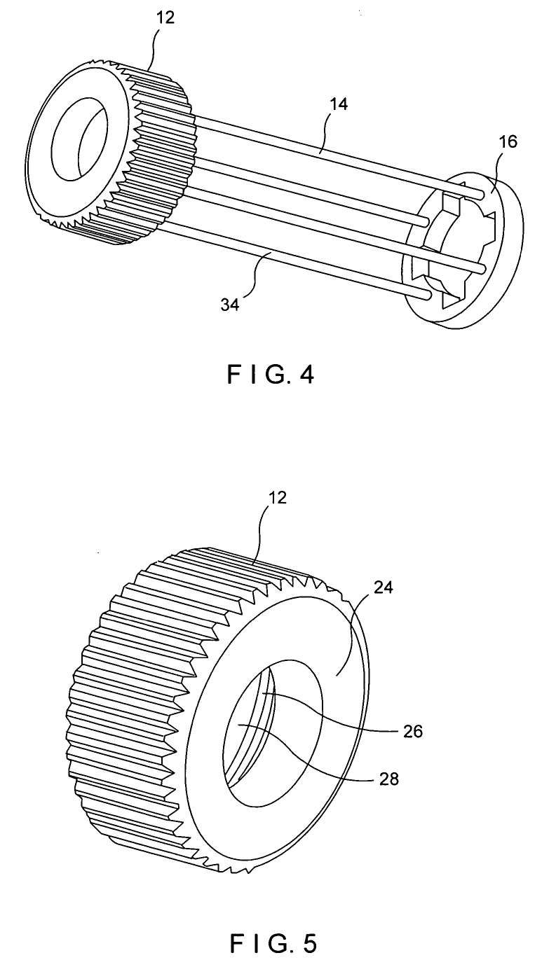

FIG. 4 is an illustrative view of the arm closing subassembly 34 shown in FIG. 2. As shown, the device lock 12 is connected to the arm closing ring 16 via the guide rods 14. The arm closing subassembly 34 is advanced by the user toward the distal end of the cervical tenaculum 2 to urge the end-effectors 6 toward the cervix. (As used herein “distal” refers to the end of the device away from a medical professional using the device, and “proximal” refers to the end of the device towards the medical professional.) More specifically, as the subassembly 34 is advanced distally, the arm closing ring 16 contacts the arms 4 drawing increasingly more distal portions of the arms 4 within its inner diameter and causing the end-effectors 6 to move radially inward to contact the cervix. The pressure applied by the end-effectors 6 against the cervix is increased as the arm closing subassembly 34 is moved further distally.

When pressure is applied by the user against the force of the cam spring 43, the cam lock 22 moves to the unlocked position so that the arm lock 10 can be advanced towards the distal end such causing the arm closing ring 16 to close the arms 4. At the same time, the main spring 45 which is attached to the arm lock 10 is pulled taught and elongated. Pressure on the cam lock 22 is then released, causing the cam lock 22 to engage with the guide rods 14, locking the arm lock 10 in position. The arms 4 can be released immediately from the closed position by pushing an actuator 41 on the cam lock 22 releasing the cam lock 22 from engagement with the guide rods 14 and allowing the main spring 45 to return to its resting position, pulling the arm lock 10 and/or the arm closing subassembly 34 proximally, thereby causing the arms 4 to spring open. Accordingly, the rapid release of the arms 4 from the locked position may be effectuated by the user, to quickly withdraw the cervical tenaculum 2 from the patient.

FIG. 5 is an illustrative detail view of the outer ring 24 of the device lock 12 of FIG. 2. The outer ring 24 fits on the proximal end of the tenaculum 2 and has an outer ring hole 28 offset from the inner ring hole 30, so that the outer ring 24 can be rotated relative to the inner ring 26 to apply a radial pressure on an inserted device 32. For example, the outer ring 24 and the inner ring 26 may rotate about a common axis which may substantially coincide with a longitudinal axis of the device. In this manner the device 32 may be locked in place within the central passage of the tenaculum 2. FIG. 6 is an illustrative view of the inner ring 26 of the device lock 12 of FIG. 2. The inner ring 26 is mounted adjacent to the outer ring 24 and has an inner ring hole 30 that is offset from the outer ring hole 28 so that the outer ring 24 can be rotated relative to the inner ring 26 to apply radial pressure as described above. The inner ring 26, for example, may have four guide rod holes 36 receiving guide rods 14.

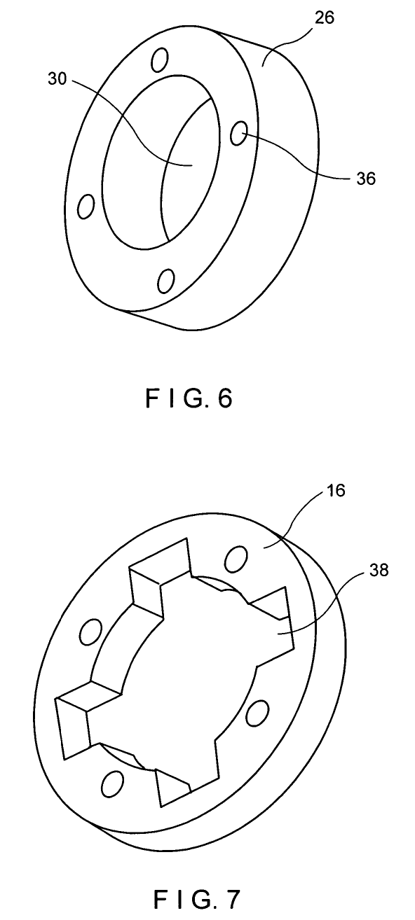

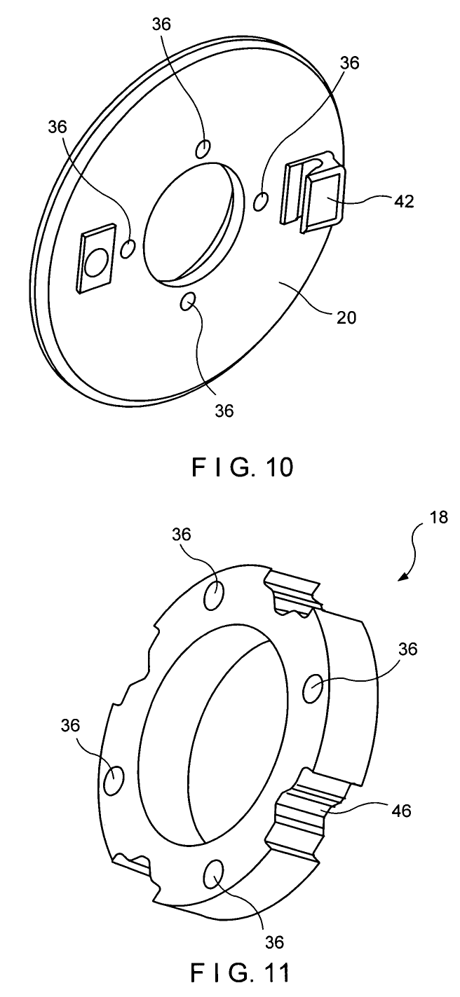

FIG. 7 is an illustrative view of the arm closing ring 16. The arm closing ring 16 may comprise a plurality of arm closing grooves 38, each of which is formed to fit a corresponding one of the arms 4. The grooves 38 also allow the arm closing ring 16 to advance forward to the distal end of the tenaculum 2 to fully close the arms 4. FIG. 8 is an illustrative view of the arm lock subassembly 40 shown in the exemplary embodiment of FIG. 2. The arm lock subassembly 40 includes a cam lock 22 mounted adjacent to the cam lock base 20 so that the cam lock 22 fits into a cam lock base groove 42 on the cam lock base 20. FIG. 9 shows an illustrative view of the cam lock 22 of FIG. 2. The cam lock 22 includes a cam lock tab 44 that fits into the cam lock base groove 42 shown in FIG. 10. The cam lock base 20 may be disposed adjacent to the cam lock 22 and may further include guide rod holes 36 each of which is adapted to receive a corresponding one of the guide rods 14. FIG. 11 shows a perspective view of the clamp arm ring 18, which fits adjacent to the cam lock base 20 and includes clamp arm grooves 46 each of which is adapted to receive a corresponding one of the arms 4. The clamp arm ring 18 further includes guide rod holes 36 each of which receives a corresponding one of the guide rods 14.

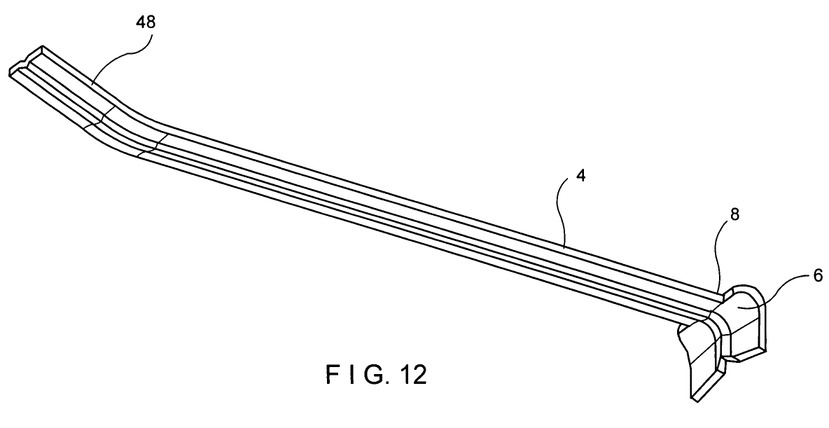

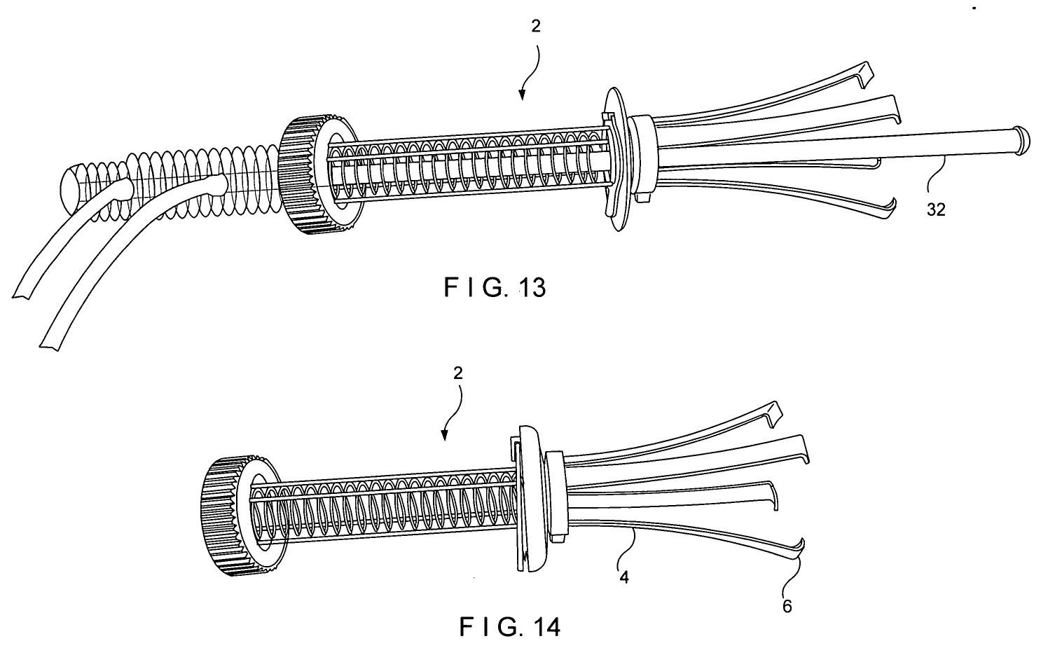

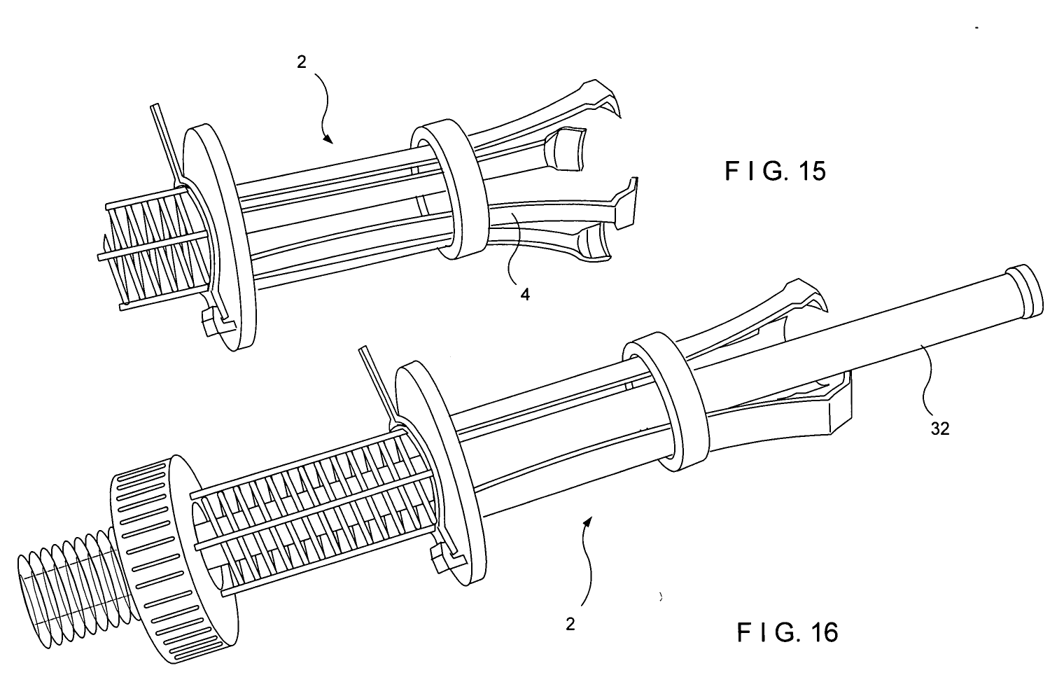

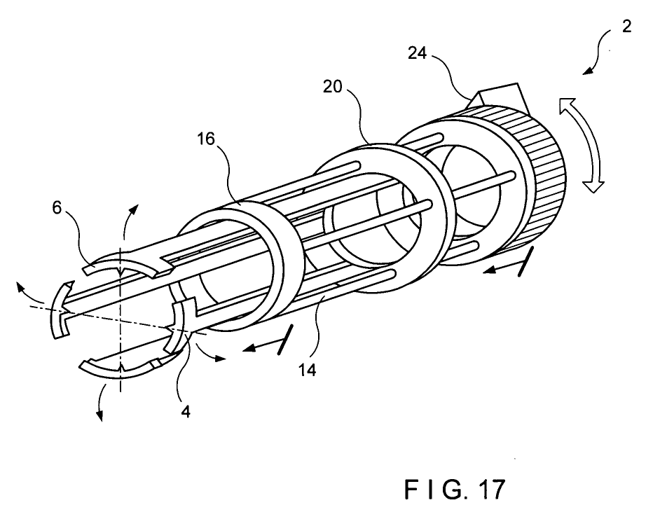

FIG. 12 is an illustrative view of an arm 4 of the exemplary tenaculum 2. As described above, the arm 4 includes an end-effector 6 at its distal end 8. The proximal end 48 of each arm 4 fits into a corresponding clamp arm groove 46 and a corresponding arm closing groove 38 of the arm lock subassembly 40, as described above. In one embodiment, the proximal ends 48 of the arms 4 are mechanically attached to an element of the arm lock 10, for example via fasteners. FIG. 13 shows an exemplary embodiment of the tenaculum 2 including a device 32, such as a sheath or a scope, inserted therethrough substantially along its longitudinal axis. FIG. 14 shows the tenaculum 2 with its arms 4 and end-effectors 6 in an open position, while FIG. 15 shows an enlargement of the arms 4. FIG. 16 shows the tenaculum 2 including an inserted device 32, with its arms 4 in the closed position, and FIG. 17 shows a perspective diagram of the tenaculum 2 with its arms 4 closed. FIG. 18 illustrates a detail of the arm lock 10 of the tenaculum 2. The arm lock 22 is biased to the locked position by a resilient element such as a cam spring 43 located between the cam lock base 20 and the actuator portion 41 of the cam lock 22.

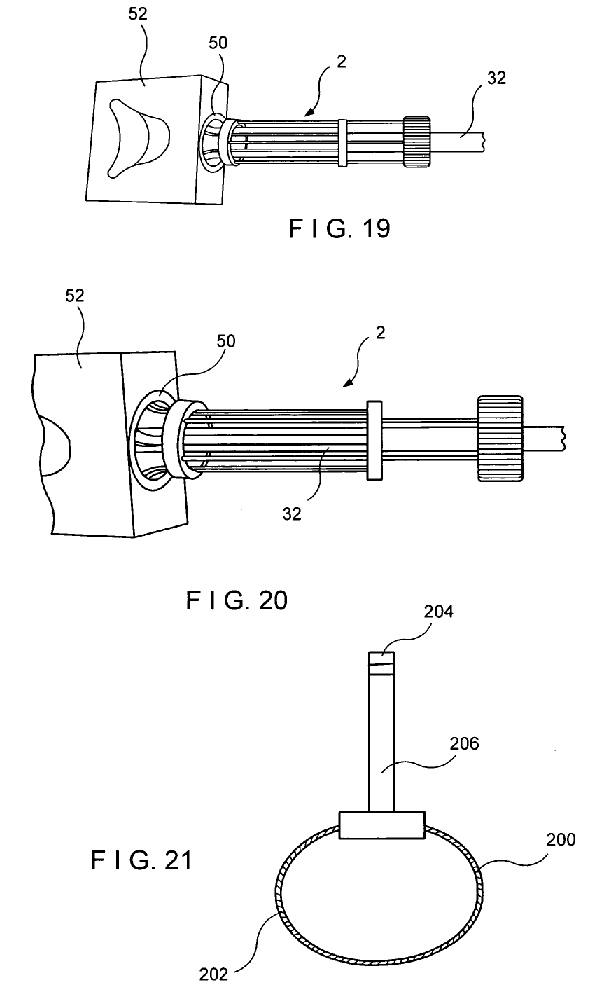

The operational use of the exemplary tenaculum 2 is described with reference to FIGS. 19 and 20, which are illustrative of the tenaculum 2 with its arms 4 and end-effectors 6 in the closed position around a simulated cervix 50. During the medical procedure, a device 32 may be inserted into the cervix 50 to access the uterus 52 of a female patient. The device 32 can be inserted before, during, or after insertion of the cervical tenaculum 2. The device lock 12 may then be rotated by the user to lock the inserted device 32 to the cervical tenaculum 2, to immobilize the inserted device 32 relative to the tenaculum 2 while the uterus 52 is assessed. As described above, sliding the arm closing subassembly 34 towards the cervix 50 causes the arms 4 and the end-effectors 6 to close and tighten around the cervix 50. The arm lock 10 may then be used to lock the arm closing assembly 34 in place to maintain the clamping force of the arms 4 and the end-effectors 6 to seal the cervix 50 about the elongated shaft of the inserted device 32. In a different embodiment, the tenaculum according to the invention may comprise substantially U-shaped components which allow the tenaculum to be placed around a device which has already been placed within the uterus. The tenaculum also may be formed from two separate halves, which can be positioned around a previously inserted device and then attached to one another to form a unitary device.

As would be understood by those skilled in the art, after the cervix 50 has been sealed around the device 32 by the pressure applied by the arms 4, medical procedures may be carried out within the uterus 52 using the inserted device 32. For example, a hot sterile solution may be injected through the inserted device 32 to ablate the endometrial lining of the uterus. The seal provided by the tenaculum 2, ensures that substantially none of the solution will leak out of the uterus during such a procedure. Alternatively, a gas may be introduced in the uterus 52 through the inserted device 32 and maintained therein by the seal.

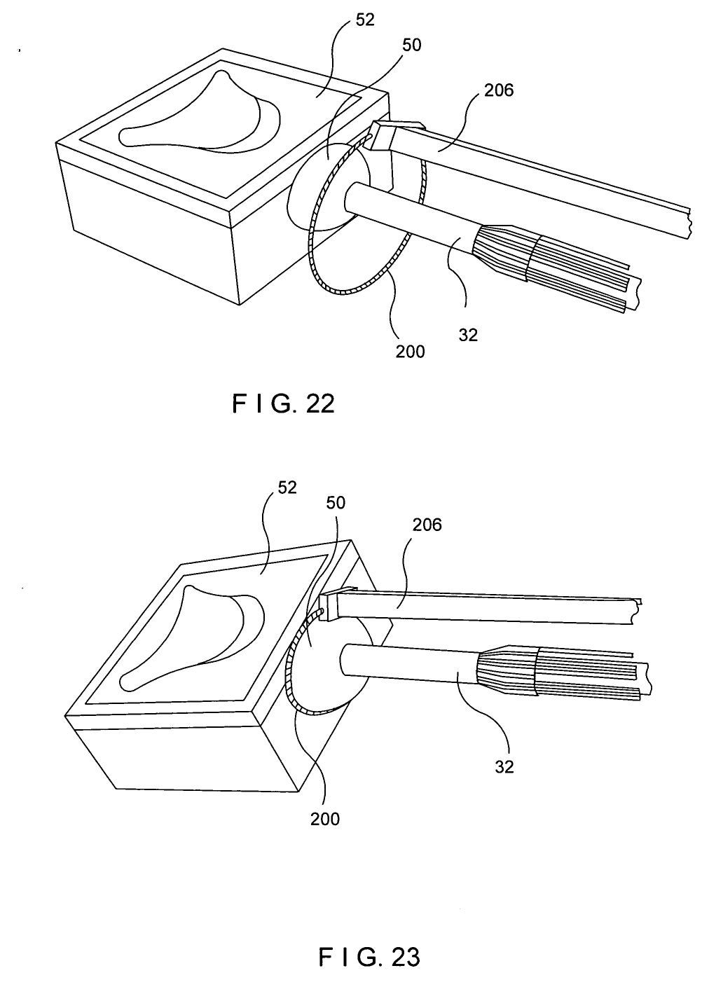

An alternate exemplary embodiment according to the present invention is described with reference to FIGS. 21-23. This embodiment of a tenaculum comprises a cervical constriction element which may, for example, be a loop 200 made of a filament such as a braided or monofilament wire having two segments, a loop segment 202 and a leg segment 204. The loop segment 202 extends through the short leg of a “T” shaped holder 206, and the leg segment 204 extends through the long leg of the “T” shaped holder 206. The T shaped holder 206 holds the loop segment 202 perpendicular to the axis of the longer leg of the T shaped holder 206, and the leg segment 204 substantially follows along this axis of the longer leg of the T shaped holder 206. During a medical procedure which is represented schematically in FIGS. 22 and 23, an inserted device 32 (e.g., a sheath) is placed in the uterus 52. The loop segment 202 is then placed over the cervix 50, and the leg segment 204 acting as a control transmittal element is pulled away from the uterus 52 to reduce the diameter of the loop segment 202. The loop segment 202 therefore tightens around the cervix 50, creating a seal between the cervix 50 and the inserted device 32. As would be understood by those skilled in the art, the T shaped holder 206 may be made of materials such as metals or plastics, which are bio-compatible and have appropriate mechanical properties.

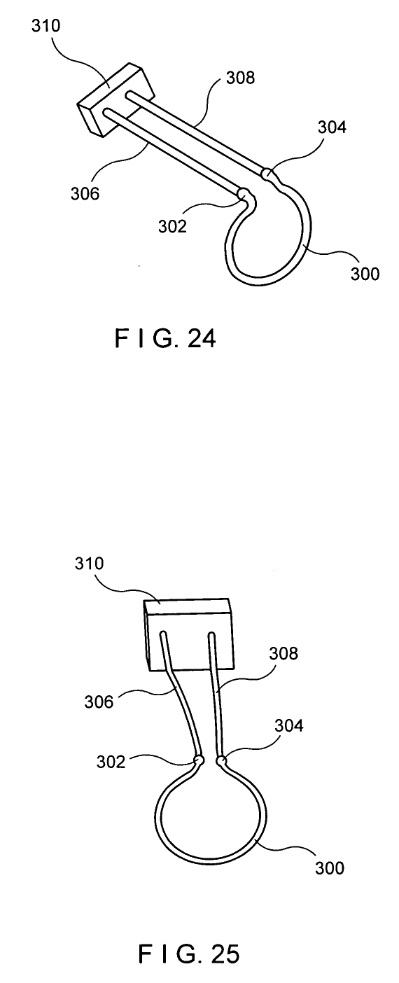

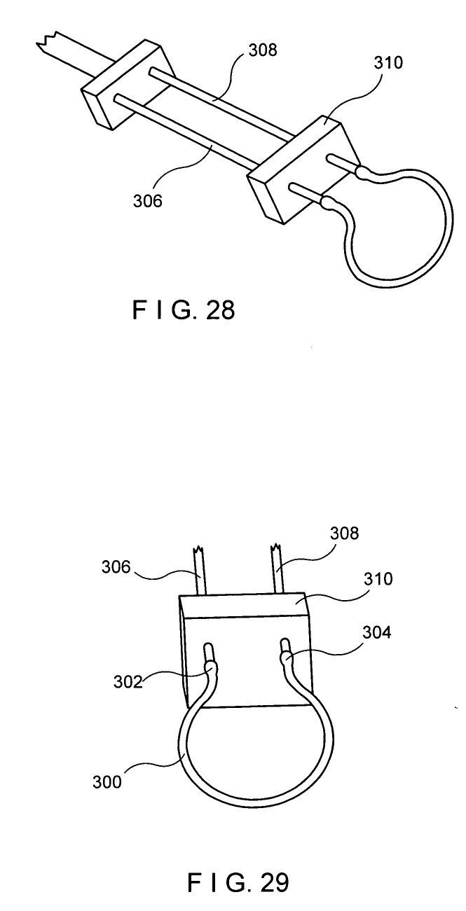

A different exemplary embodiment of the cervical tenaculum according to the invention is presented in FIGS. 24-29. In this embodiment, a substantially planar coil 300 is shown attached to two rods 306, 308 disposed at the ends 302, 304 of the coil 300. A block 310 is adapted to slide along the rods 306, 308 and is shown in a ‘closed’ configuration in FIGS. 24 and 25 wherein the ends 302, 304 of the coil 300 are adjacent to one another. The coil 300 which may be for example a spring, is biased to maintain this ‘closed’ configuration. However, when the block 310 is moved by the user toward the coil 300, the bowed shape of the rods 306, 308 causes the ends 302, 304 to spread apart into a second, ‘open’ configuration as shown in FIGS. 28 and 29. The block 310 maintains the ends 302, 304 in this ‘open’ configuration against the tendency of the coil 300 to spring back into the ‘closed’ configuration. The rods 306, 308 and the coil 300 are preferably constructed from a metal, plastic or ceramic that has strong shape memory properties or from any other suitable material having such shape memory properties. The material will also be selected to have appropriate mechanical properties to provide the desired clamping force to the cervix. In various exemplary embodiments, the coil 300 is designed to complete between about 350 degrees and about 540 degrees of revolution when wrapped around the cervix of a patient. The block 310 can be made for example from either metals or plastics.

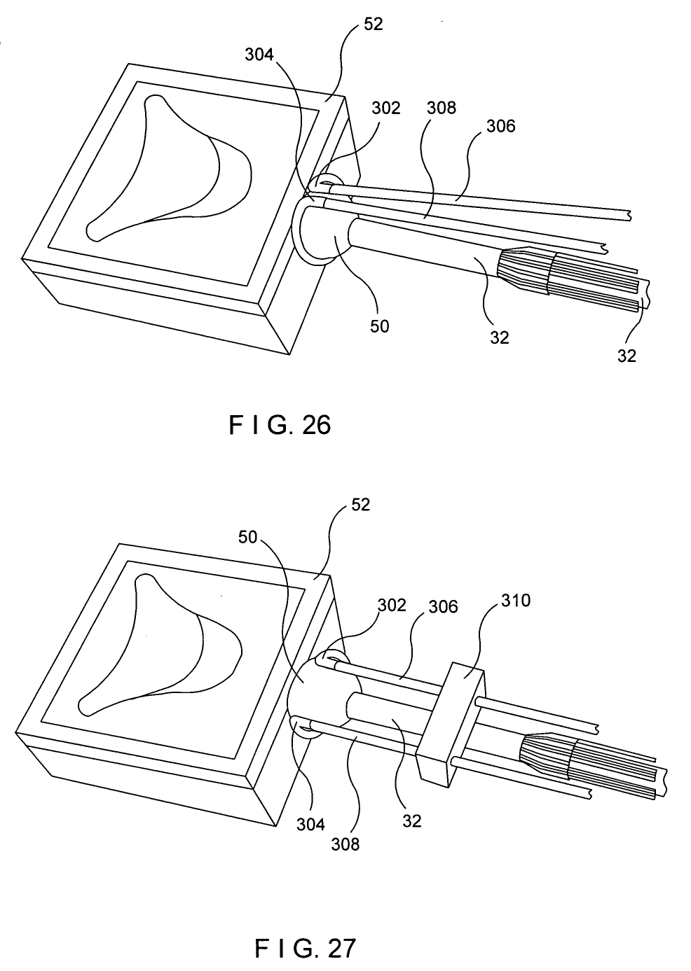

During a medical procedure, the block 310 is moved toward the coil 300 to bring the coil 300 to the ‘open’ configuration. The open coil 300 is placed over the cervix 50, as shown in FIG. 27 and the block 310 is moved away from the coil 300 to bring the coil 300 to the ‘closed’ configuration, as shown in FIG. 26. When closed, the coil 300 applies pressure to the cervix 50, sealing the cervix 50 around an inserted device 32 which has been introduced therethrough into uterus 52. Alternate designs may be made in which the coil 300 is replaced by a spring with an essentially round profile when viewed from the end. Such springs can be produced, for example, from spring steel or 400 series hardened stainless steel with a configuration spanning from about 365 degrees of arc to about a 539 degree of arc.

A cam rod extending axially to a cam block may protrude from each end of the coil wire. The cam block may contain a pair of holes or one oblong hole/slot that slidably receives the pair of cam rod. As the cam is advanced towards the coil, the cam arms are drawn together resulting in the spring coil “opening” to result in an effectively larger frontal diameter that can be placed over the cervix. When the block is retracted, the spring coil returns to its normal ‘closed’ position, exerting a radial closure force on the cervical opening. Alternatively, in another embodiment, the spring coil may exceed about 540 degrees of rotation. As the cam block is advanced the coil becomes smaller, relying on the cam position to secure the tissue compression. In another alternative embodiment, a coil spanning about 360 degrees is provided. The cam arms running axially from the coil ends cross each other so that the cam block motion now operates in the opposite direction so that the user pulls back on the block to open the coil.

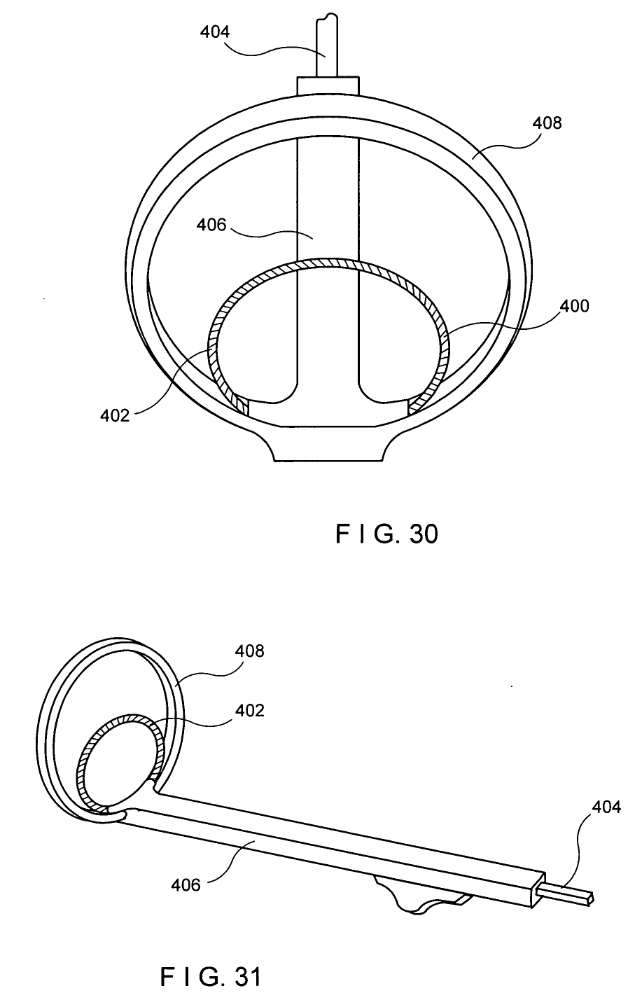

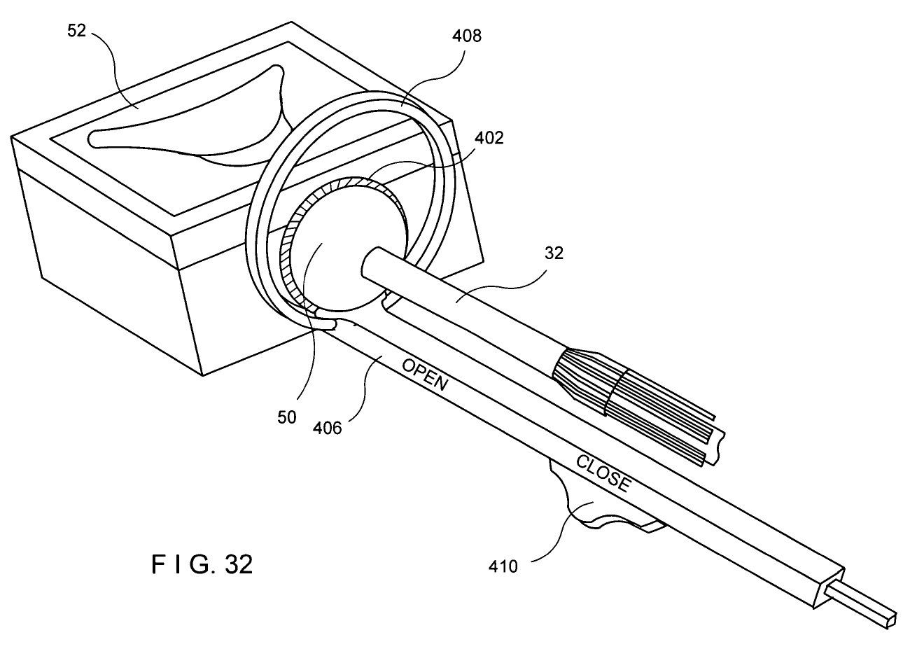

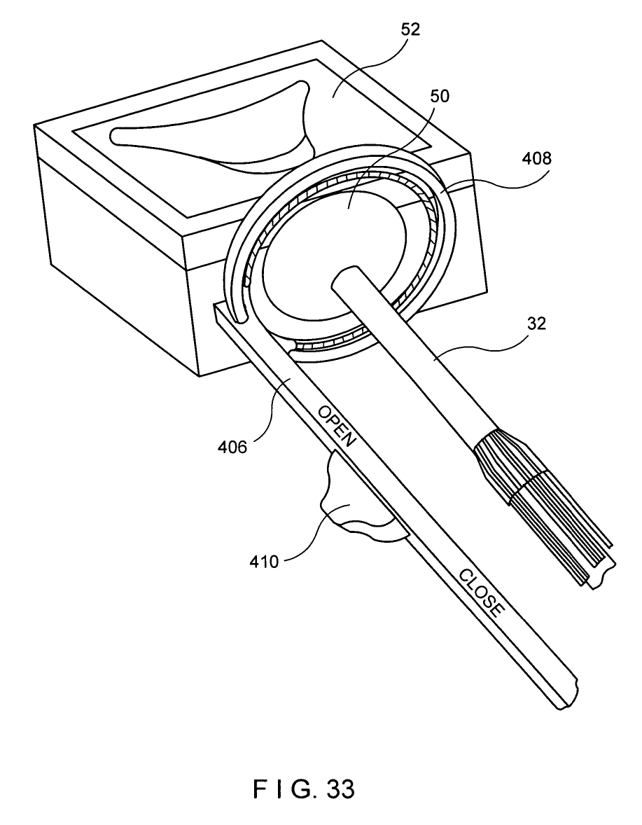

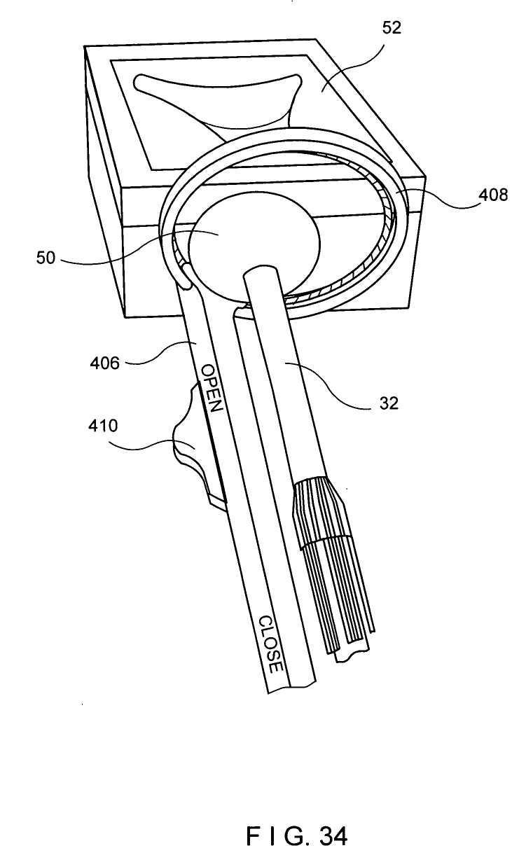

Referring to FIGS. 30-34, another exemplary embodiment of a tenaculum comprises a loop 400 which is preferably made of a filament such as a braided or monofilament wire having two segments: a loop segment 402 and a leg segment 404. The loop segment 402 extends through the short leg of a “T” shaped holder 406, and the leg segment 404 extends through the long leg of the “T” shaped holder 406. The “T” shaped holder 406 holds the loop segment 402 substantially perpendicular to an axis of the longer leg of the “T” shaped holder 406, and the leg segment 404 generally follows along this axis of the long leg of the “T” shaped holder 406. Additionally, a guide ring 408 may be used to house the loop segment 402. In operation, as shown in FIGS. 33 and 34, a device 32 is inserted into the uterus 52. The guide ring 408, which houses the loop segment 402, is placed over the cervix 50 and against the uterus 52. The guide ring 408 ensures that the loop segment 402 remains open and substantially perpendicular to the axis of the longer leg of the “T” holder 406 during placement of the device.

As shown in FIG. 32, once over the cervix 50, a slide tab 410 attached to the leg segment 404 is moved away from the uterus 52 to reduce the diameter of the loop segment 402. The “open” position of the slide tab is located towards the end of the device near the loop 400, while the “closed” position is located away from the end of the device adjacent to the loop 400. Thus, moving the slide tab 410 away from the uterus 52 tightens the loop segment 402 around the cervix 50 to create a seal between the cervix 50 and the inserted device 32 (or other medical instrument).

In one embodiment, a clamp for sealing a body structure may include at least two arms arranged concentrically about a first axis and extending along the device. Also included are an end having a first profile, a collar slidably engaged with the at least two arms such that sliding the collar moves the at least two arms towards the first axis and at least two guide rods each extending through a hole on a ring adjacent to the collar. The clamp may also include a first lock biased to a first position and movable to a second position, wherein the ring contacts at least one of the guide rods in the first position. In this embodiment, the clamp defines a space extending along the first axis for receiving a sheath, a scope or other medical device. The clamp is manually coupled to the sheath, scope or other medical device or coupled thereto using a spring.

In another embodiment, a clamp for sealing a body structure includes a substantially planar spring having a first end and a second end, and which completes at least about one revolution about a first axis. The spring may be biased towards a first, closed position such that the first end and second end are located adjacent to each other when the spring is in the first position. First and second rods, each generally aligned with the first axis, are included with the first rod connected to the first end and the second rod connected to the second end. Thus, moving the first and second rods apart moves the ends of the spring to a second, open position. In the second position the first end and second end are located further apart than in the first position. The rods can be bowed or bent such that they move further apart in the second position.

In yet another embodiment, a clamp for sealing a body structure includes a filament including a first segment forming a loop and a second segment extending along a first axis, wherein the loop is substantially perpendicular to the first axis, and pulling the first segment away from the loop reduces the diameter of the loop. Additionally, this embodiment may include a pusher ring housing the loop and providing pushability to protrude the cervix through the ring lumen. This embodiment may also include a moveable loop adapted to circumvent the cervix and to provide compression to the uterine muscle. The loop may move independently from the ring and can be manipulated by drawing at least one end of the loop wire.

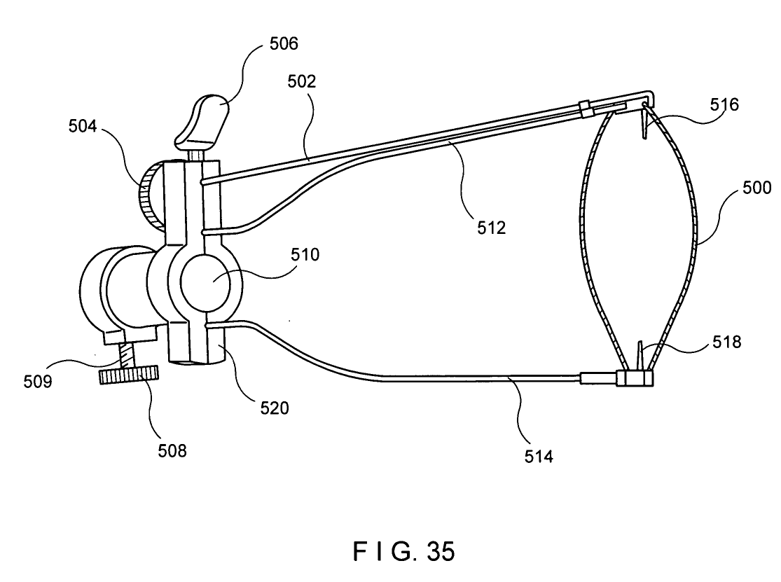

FIGS. 35-37 depict an additional embodiment of a cervical tenaculum comprising both a loop 500 and two spikes 516, 518 disposed on control arms 512, 514. Additional or fewer spikes and control arms may be used, as will be apparent to those of skill in the art. The loop 500 may extend through apertures at the ends of the control arms 512, 514. A pull wire 502 may be used to connect to one end of the loop 500, to extend along one of the control arms 512 through a pull wire lock 506 located in a base 520, and to reach a pull knob 504. The loop 500 and/or the pull wire 502 may, for example, be formed from a braided or monofilament wire. The control arms 516, 518 are preferably attached to the base 520 while a bore 510 extends through the base 520. A bore lock 508 is located at one end of the bore 520 and may include a screw 509 threaded therethrough. The end of the screw 509, when tightened, extends through the bore lock 508 and contacts a medical device 32 disposed within the bore 510, substantially immobilizing it. In other embodiments, the bore lock 508 may take other configurations for locking a medical device in the bore of a cervical tenaculum, as will be appreciated by those of skill in the art.

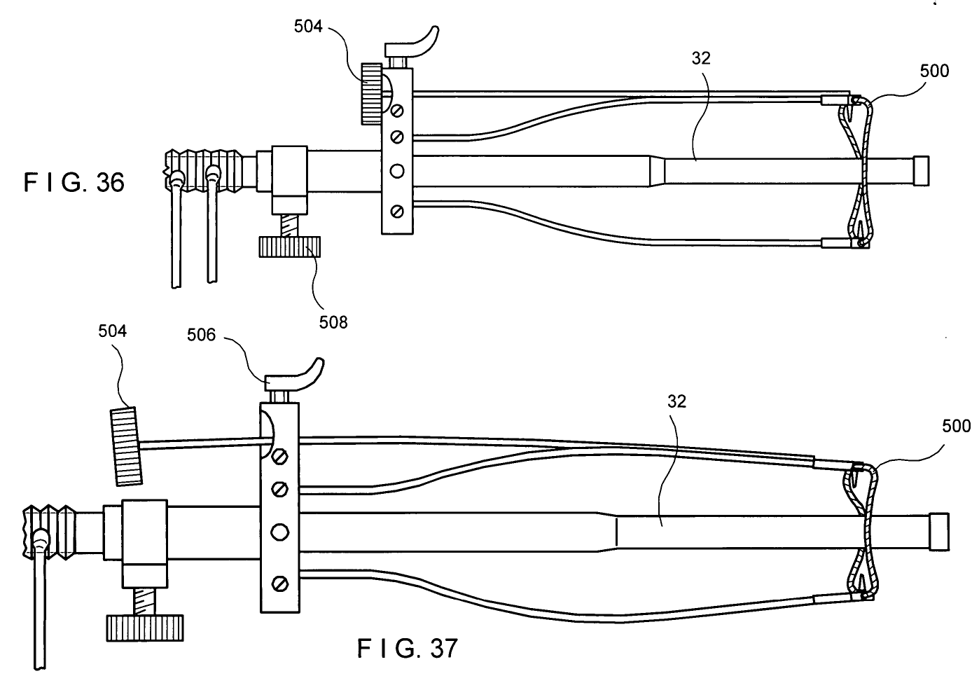

As would be understood by those skilled in the art, the loop 500 may be placed in an open or a closed configuration during execution of a medical procedure. When the loop 500 is in the open configuration, the pull knob 504 is positioned against the base 520 and the diameter of the loop 500 is at its largest, as seen in FIG. 36. When the pull knob 504 is drawn away from the base, the pull wire 502 attached to the loop 500 is pulled, decreasing the size of the loop 500 (as seen in FIG. 37) and placing the device in a closed configuration. The loop 500 may then be locked in place with the pull wire lock 506. Since the control arms 512, 514 are biased toward the open position, the pull wire lock 506 is adapted to prevent the control arms 512, 514 from spreading apart and pulling the pull knob 504 towards the base 520 to increase the diameter of the loop 500.

When using the tenaculum during a medical procedure, a medical device 32 is inserted through the bore 510 and locked in place. Then, the loop 500 is moved to an open position, the loop 500 is placed around the cervix and the medical device 32 is inserted through the cervix into the uterus. The medical professional then pulls on the pull knob 504, to place the device in the closed configuration with the loop 500 tightened around the patient's cervix. The loop 500 applies pressure around the entire periphery of the cervix, sealing the cervix against the medical device 32 inserted through the cervix into the uterus. Additionally, the exemplary spikes 516, 518 engage the cervix when the loop 500 is in a closed configuration, helping to prevent the loop 500 from sliding off of the cervix. The exemplary device provides additional traction with the cervix, since it is otherwise difficult to put spikes on the loop itself such that they are properly oriented to engage the cervix when the loop is constricted.

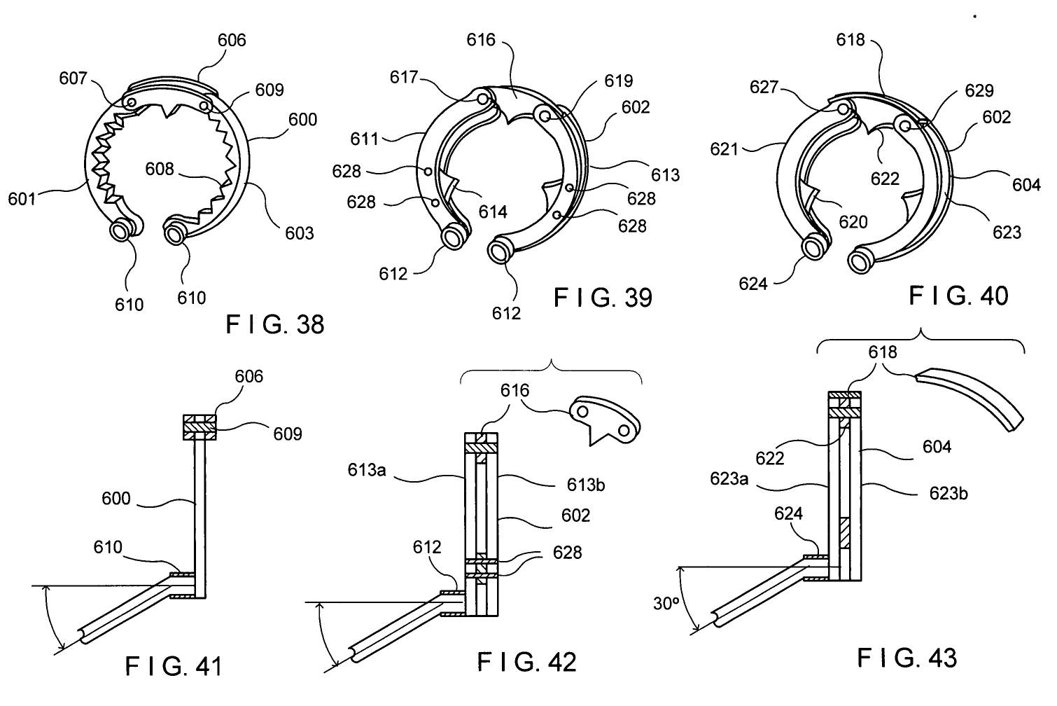

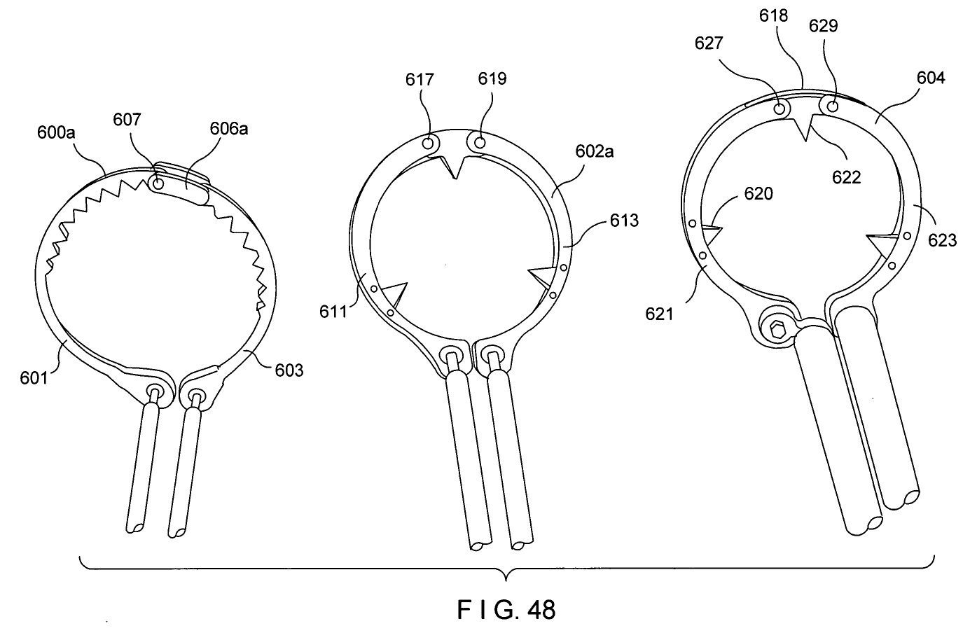

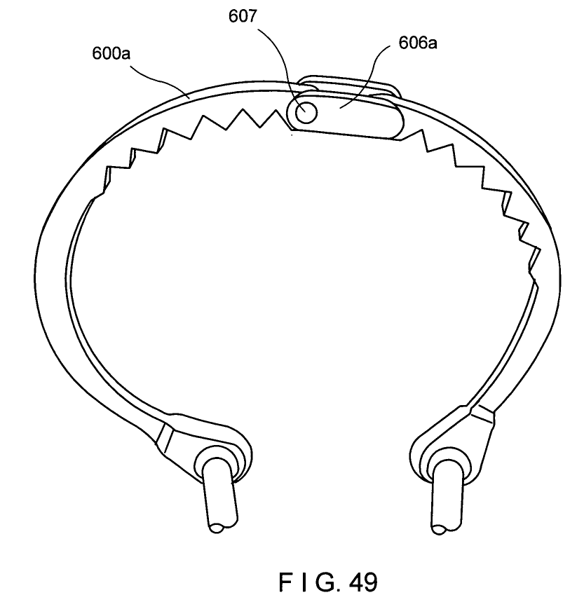

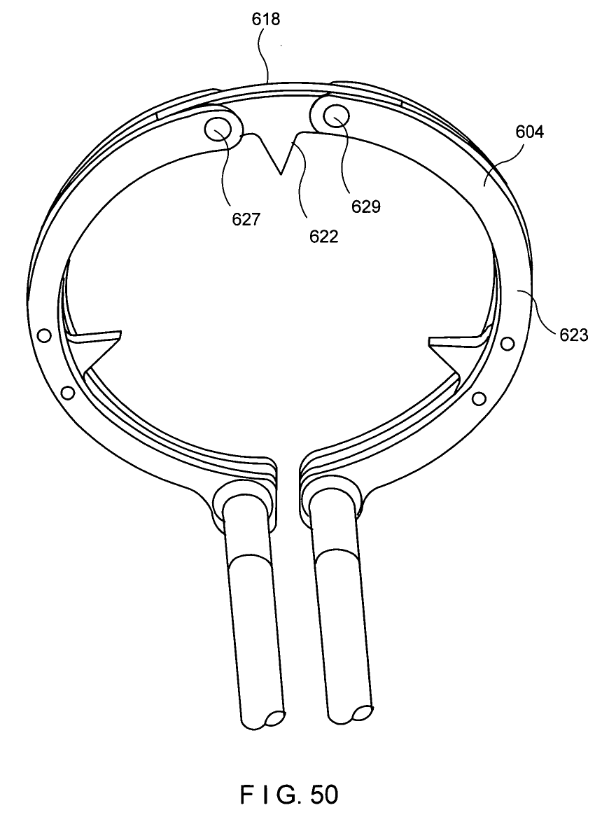

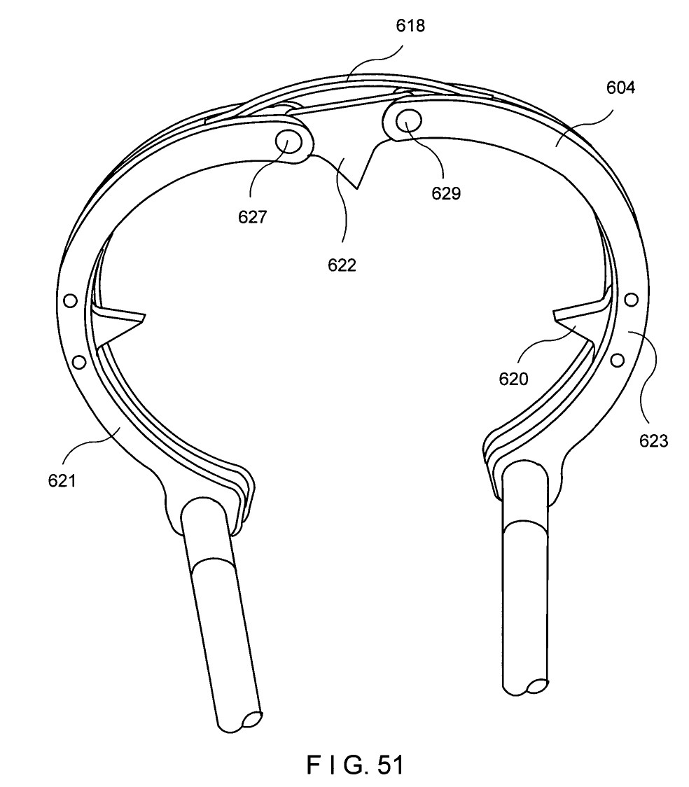

FIGS. 38, 41, 46, 47, 48, 49, and 52 show an embodiment of a cervical tenaculum that comprises a clamp 600. In these figures, certain non-limiting examples of dimensions of the devices are given, which may be modified as will be understood by those of skill in the art. The clamp 600 comprises two arcs 601, 603 that may be connected via a link 606. The link 606 preferably includes a spike and each arc 601, 603 preferably includes one or more teeth 608 on an inner curve thereof. At the end of each arc 601, 603, remote from the link 606, there is an attachment point 610 for a medical clamp 626, for example, a hemostat or forceps. The medical clamp 626 may also be integral with the clamp 600. The medical clamp 626, when attached to the clamp 600, allows the clamp 600 to be opened and closed around the cervix thus applying a sealing pressure to the cervix against a medical device inserted therein. As shown in the side section view of FIG. 41, the link 606 may be disposed on either side of the arcs 601, 603, such that two pins 607, 609, one on each end of the link 606, hold the link to the arcs 601, 603.

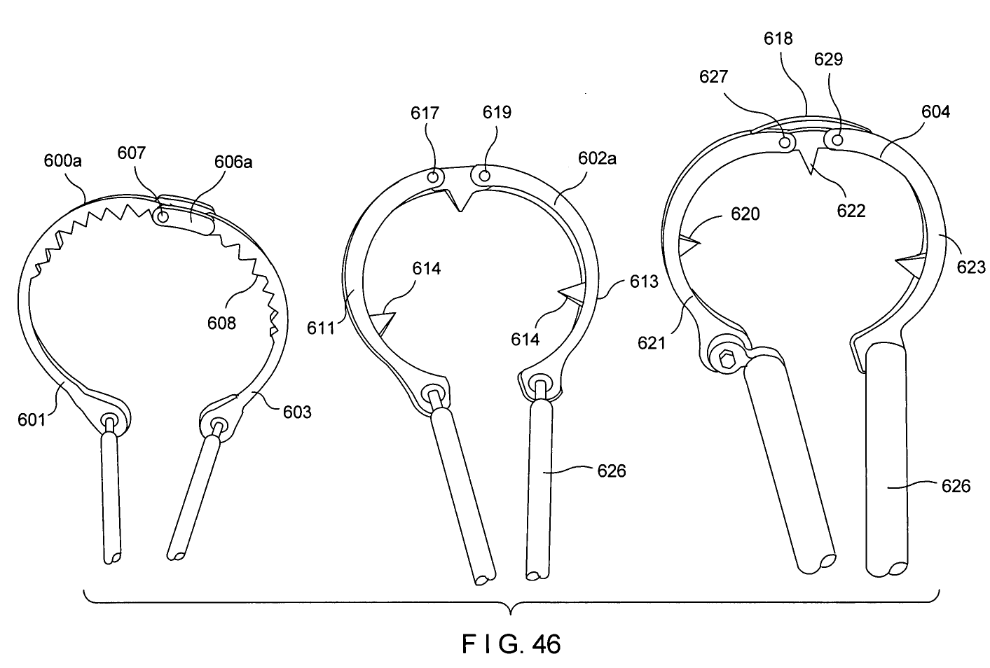

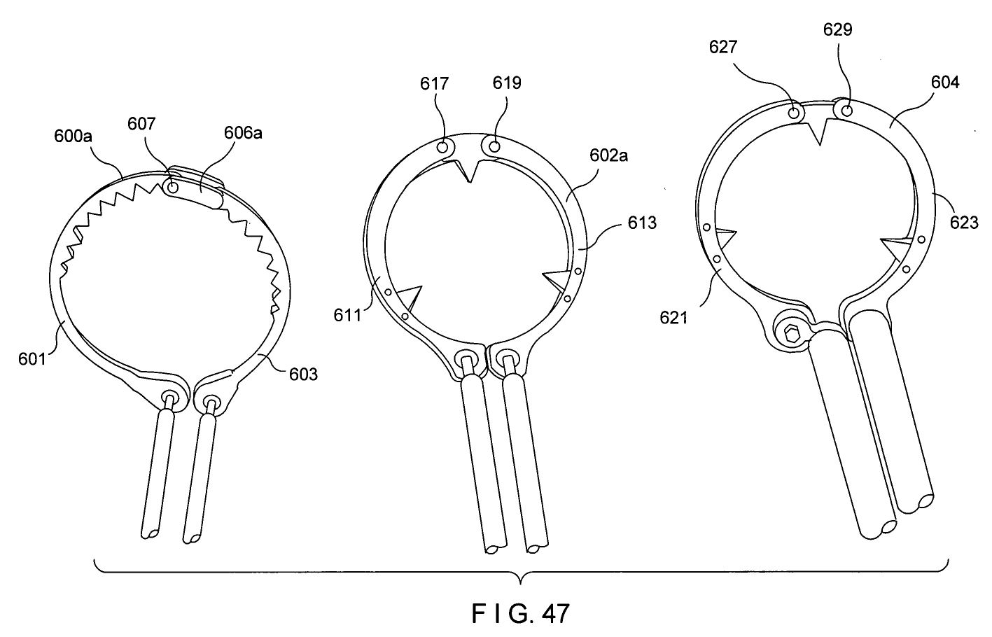

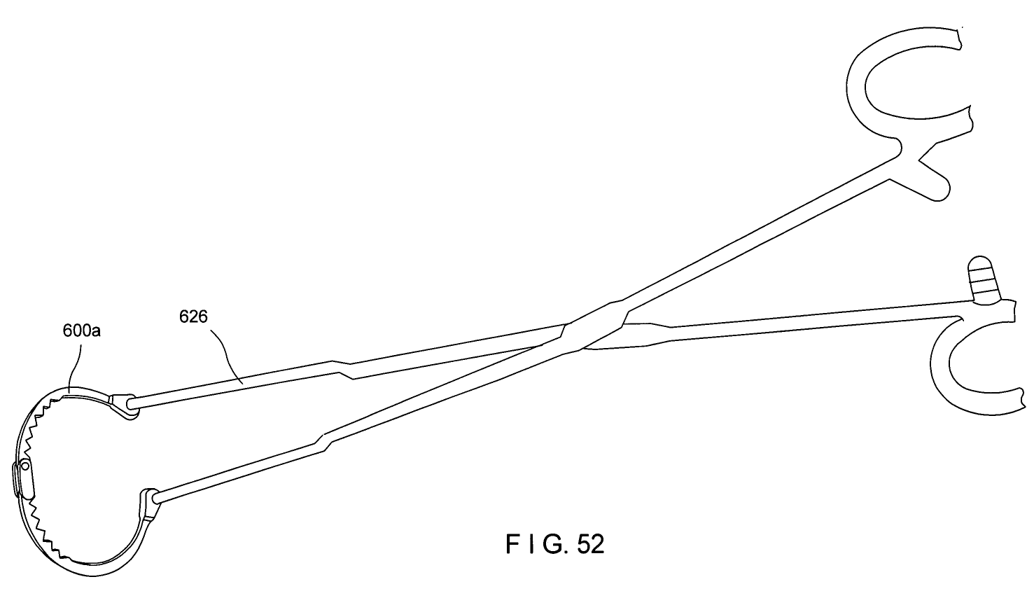

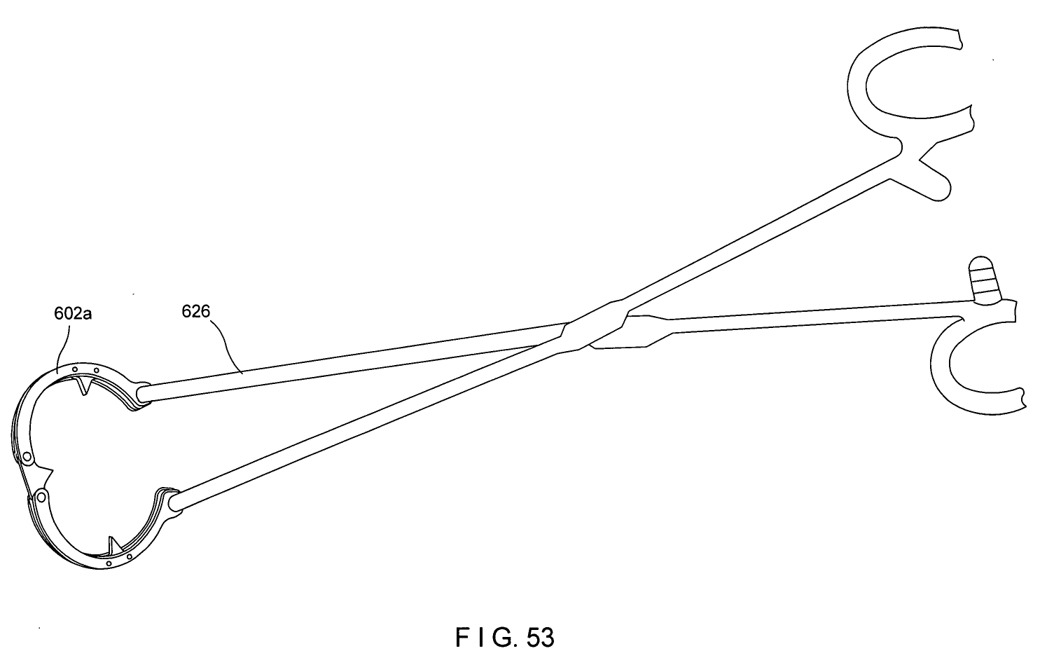

Another embodiment according to the present invention is shown in FIGS. 46, 47,48, 49, and 52. In this embodiment, the clamp 600 preferably comprises only one pin 607. Accordingly, the link 606a is affixed to a first one of the arcs 603 at a one of its ends, while the pin 607 at the other end of the link 606a allows the second arc 601 to swing between the open and closed configurations as the medical clamp 626 moves the ends of the arcs 601, 603 towards or away from each other. As shown in FIGS. 46 and 52, when the ends of the arcs 601, 603 are away from each other, the clamp 600a is in the open configuration and, when the ends are close to each other, the clamp 600a is in the closed configuration as shown in FIG. 47. Those skilled in the art will understand that, when the clamp 600a is in the closed configuration, the clamp 626 may be locked using, for example, a ratchet lock. Locking the medical clamp 626 also locks the attached clamp 600a in the closed configuration.

During use, the clamp 600, 600a is advanced in the open configuration over the cervix and a medical device is inserted through the cervix into the uterus. The medical clamp 626 is then moved to close the clamp 600 (or 600a) around the cervix. The clamp 600 applies pressure around the entire perimeter of the cervix and against and around the medical device inserted therethrough, creating a seal between the cervix and the medical device. Using the lock 625 on the clamp 626, the clamp 600 may then be locked closed. Alternatively, the clamp 600 may comprise a dedicated lock. The teeth 608 on the clamp 600 engage the cervix to help prevent slippage relative thereto. As would be understood by those skilled in the art, all or part of the clamp 600, 600a may be made from any metal compatible with surgical techniques, such as stainless steel, or from a suitable plastic.

FIGS. 39, 42, 46, 47, 48 and 53 show another exemplary embodiment of a cervical tenaculum that comprises a clamp 602. The clamp 602 has two arcs 611, 613 are connected by a link 616. The link 616 may include on each arc 611, 613 one or more spikes 614 extending from an inner curve of the corresponding arc. At the end of each arc 611, 613, remote from the link 616, there may be an attachment point 612 for a clamp 626 such as a hemostat or forceps. The clamp 626, when attached to the clamp 602, allows the tenaculum to be opened and closed around the cervix to apply a sealing pressure against a medical device extending through the cervix. As shown in FIG. 42, each of the arcs 613 may be made of two or more thin arc layers (613a and 613b are shown) of substantially similar curvature with the arc layers held together at one end by pins 628. The pins 628 may also hold the spikes 614, which are located between each of the two thin arc layers. The link 616 may be disposed between the two thin arc layers making up the arcs 611, 613 on each of its ends.

In the embodiment shown in FIGS. 46, 47, 48, and 53, the clamp 602a does not include pins holding the spikes to each of the pairs of thin arc layers that make up the arcs 611. As the link 616 is attached to each of the arcs 611, 613 at each one of its ends with a pin 617, 619, each arc 611, 613 may swing separately at the point at which the pins 617, 619 are located in order to take on the open or closed configuration. The medical clamp 626 may be used to move the ends of the arcs 611, 613 towards each other and away from the link 616, or away from each other. When the ends are away from each other, the clamp 602, 602a is in the open configuration, and when the ends are close to each other, the clamp 602, 602a is in the closed configuration. When the clamp 602 is in the closed configuration, the clamp 626 may also be locked as described above, for example, with a ratchet lock 625 on the handle. This procedure locks the clamp 602 in the closed configuration.

As with the previously described procedures, the clamp 602, 602a is advanced in the open configuration over the cervix and a medical device is inserted through the cervix into the uterus. Then, the clamp 626 is moved to close the clamp 602 (or 602a) around the cervix, applying pressure around the entire cervix and against and around the medical device inserted therethrough to create a seal between the cervix and the medical device. Using the lock 625 on the medical clamp 626, the clamp 602 is locked in position. Alternatively, the clamp 602, 602a may have its own lock. As described above, the spikes 614 formed on the clamp 602 engage the cervix to help prevent slippage. All or part of the clamp 602, 602a can be made from a metal compatible with surgical techniques, such as stainless steel, or from a suitable plastic.

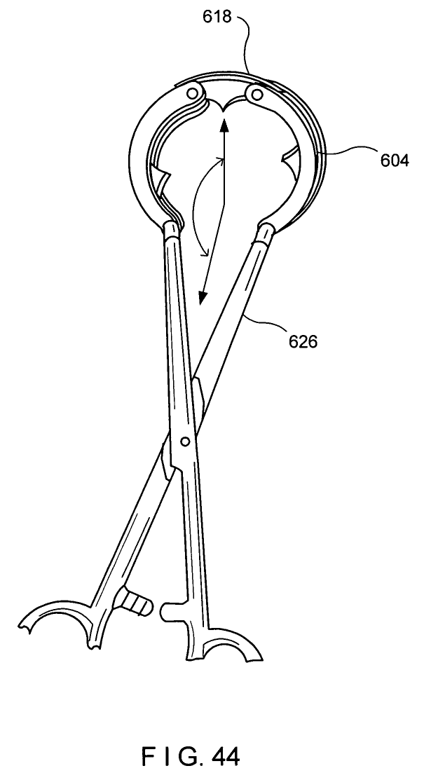

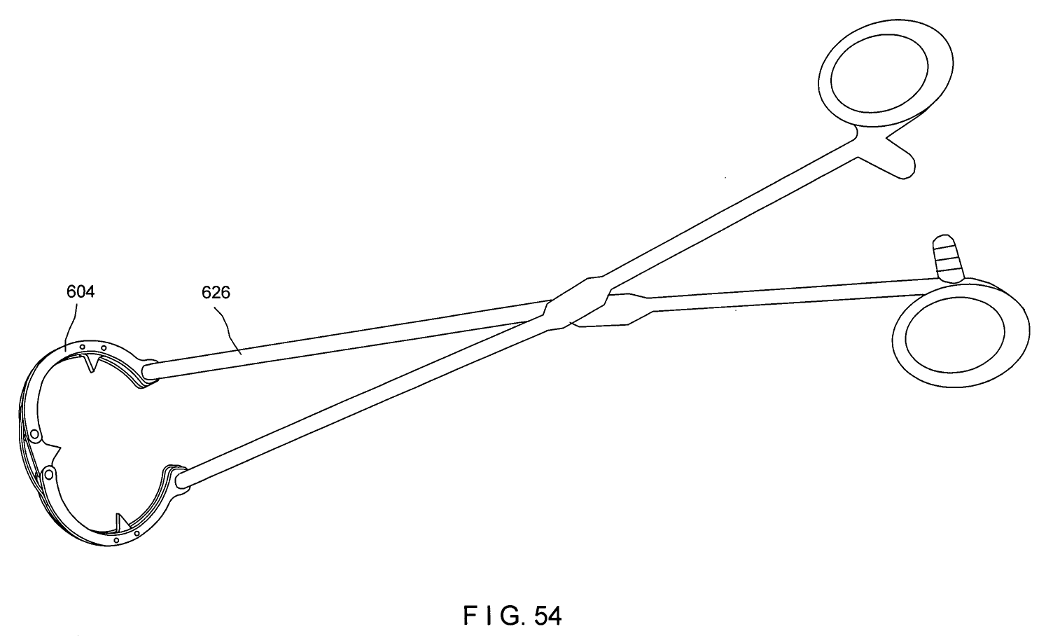

Now referring to FIGS. 40, 43, 44, 46, 47, 48, 50, 51, and 54, an embodiment of a cervical tenaculum comprising a clamp 604 is shown. The clamp 604 has two arcs 621, 623 connected by a link 622. The link 622 preferably includes one or more spikes with each arc 621, 623 one or more spikes 620 on an inner curve thereof. At the end of each arc 621, 623, an attachment point 624 for a medical clamp 626, such as a hemostat or forceps, may be provided. The medical clamp 626, when attached to the clamp 604, allows the clamp 604 to be opened and closed around the cervix, applying sealing pressure against a medical device within the cervix. As shown in the side elevation view of FIG. 43, each of the arcs, e.g., arc 623, may be made of two thin arcs (623a and 623b) of substantially the same curvature. The spikes 620 may be located between each of the two thin arcs. The link 622 may be disposed between the two thin arcs on each of its ends. Pins 627, 629 hold each end of the link 622 to each pair of the thin arcs that make up each of the arcs 621, 623 with a resilient member (e.g., leaf spring 618) attached to each of the arcs 621, 623, across the top of the link 622. The link 622 may be affixed to each of the arcs 621, 623 at each of its ends with a pin 627, 629, so that each arc 621, 623 can swing separately when the medical clamp 626 is used to move the ends of the arcs remote from the link 622.

As the arcs 621, 623 move towards or away from each other, they assume the closed and open configurations, respectively. However, the leaf spring 618 extending between the two arcs 621, 623 biases the clamp 604 toward the open configuration with both of the arcs 621, 623 moving the same distance to open the clamp 604 uniformly. When the ends of each of the arcs 621, 623 are away from each other, the clamp 604 is in the open configuration, and when the ends of each of the arcs 621, 623 are close to each other the clamp 604 is in the closed configuration. The open position, with the leaf spring 618 bowed, is shown in FIGS. 46 and 51. When the clamp 604 is in the closed configuration, the medical clamp 626 may be locked as described above. The use of the clamp 604 according to the present exemplary embodiment is similar to that described above. Similarly to the above-described embodiments, all or part of the clamp 604 may be made from a metal compatible with surgical techniques, such as stainless steel, from any suitable plastic material or from a combination of any of these materials.

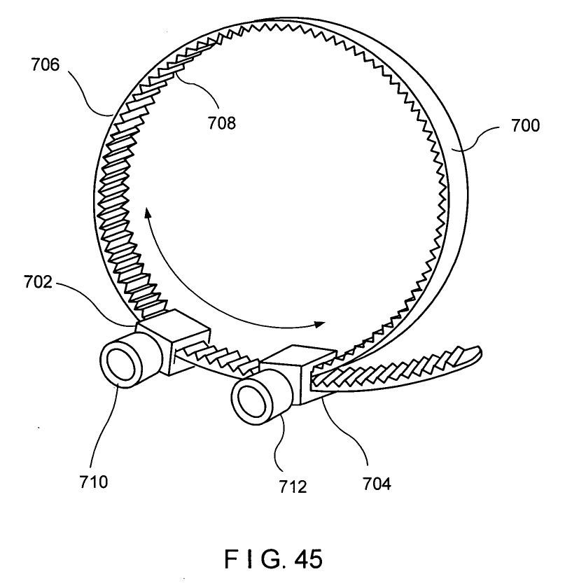

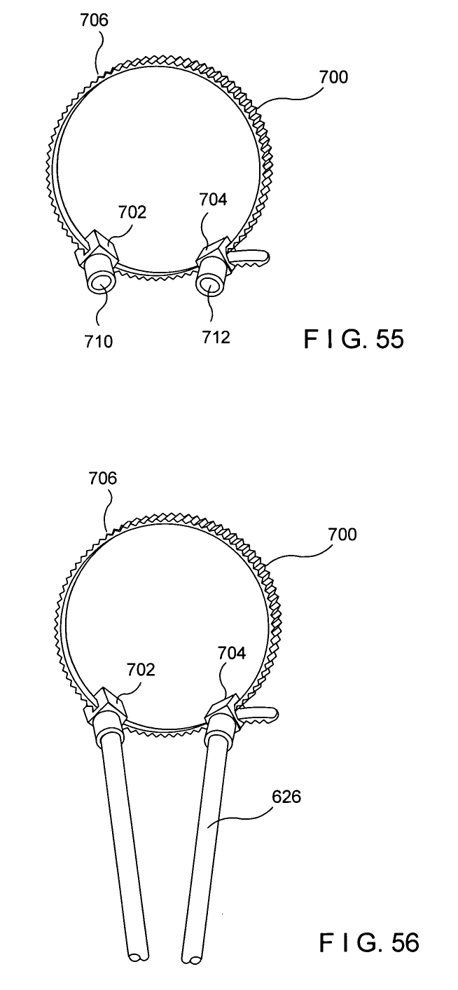

FIGS. 45 and 55-58 show a different exemplary embodiment of a cervical tenaculum according to the present invention, which comprises a tie 700 including a band 706 with a plurality of teeth 708 disposed thereon. A stationary ratchet 704 may be located at one end of the band 706 with a moveable ratchet 702 located along the band 706 which extends through the ratchet 702. The ratchets 702, 704 define apertures 710, 712, respectively, adapted to accept a medical clamp 626, such as a hemostat or forceps. As in the previously described embodiments, the exemplary tie clamp 700 may be integral with the medical clamp.

Furthermore, one or both ratchets 702, 704 may comprise a small, internal cantilever element which engages the teeth 708. The moveable ratchet 702 is designed so that it may be moved over the band 706 away from the stationary ratchet 704 (i.e., the cantilever element of the movable ratchet 702 moves over the teeth 708 of the band 706). When the moveable ratchet 702 is moved towards the stationary ratchet 704, the band 706 is pushed through the stationary ratchet 704 (i.e., the cantilever element of the movable ratchet 702 cannot move over the teeth 708 of the band 706 and pushes against them). The cantilever in the stationary ratchet 704 prevents the band 706 from being pulled back through the stationary ratchet 704 in the opposite direction. Accordingly, when the moveable ratchet 702 is moved toward the stationary ratchet 704, the diameter of the band 706 becomes smaller. This action allows the band 706 to provide a clamping force to the cervix such that it seals around a medical device inserted therethrough. The medical clamp 626 may be inserted into the apertures 710, 712 in the ratchets 702, 704, and the moveable ratchet 702 can be moved by closing the arms of the medical clamp 626.

During use, the tie 700 is advanced in the open configuration (i.e., not yet tightened) over the cervix and a medical device is inserted through the cervix into the uterus. The medical clamp 626 is then manipulated to move the moveable ratchet 702 toward the stationary ratchet 704 to decrease the diameter of the band 706 and close the tie 700 around the cervix causing the tie 700 to apply pressure around the perimeter of the cervix and against and around the medical device inserted therethrough to create a seal between the cervix and the medical device.

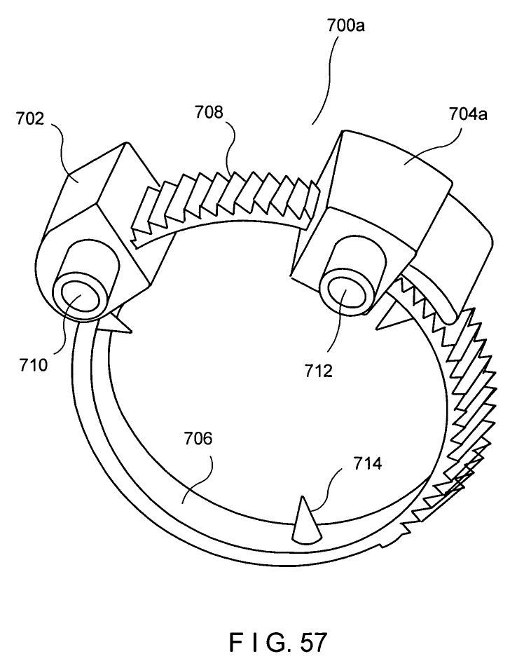

A different exemplary embodiment of a tie-shaped cervical tenaculum according to the invention is shown in FIG. 57. In this case, a tie 700a comprises a stationary ratchet 704a comprising one or more spikes 714. The tenaculum 700a according to the present embodiment operates to tighten around the cervix substantially in the same manner as the tie 700 described above with the spikes 717 adapted to engage the cervix to help prevent slippage. However, the exemplary tenaculum tie 700a comprises a stationary ratchet 704a that may be squeezed by the user so that the cantilever member within the stationary ratchet 704a disengages from the teeth 708 on the band 706. The teeth 708 may cover a large portion of the band 706, as required for certain medical procedures. Accordingly, the diameter of the band 706 can be enlarged by sliding the band 706 toward the moveable ratchet 702, for example to rapidly release the constricted band 706 from the cervix. Other different embodiments of the tie tenaculum which don't include a releasable lock can be cut to release them from the cervix.

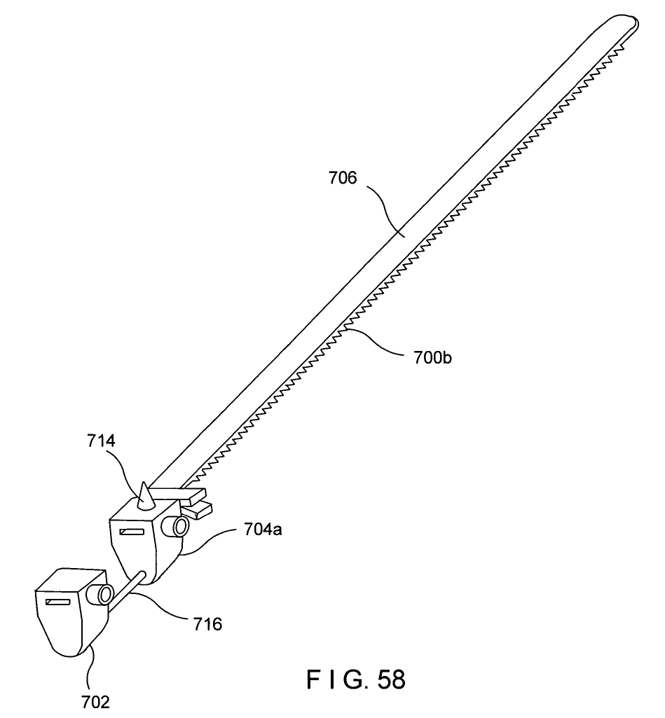

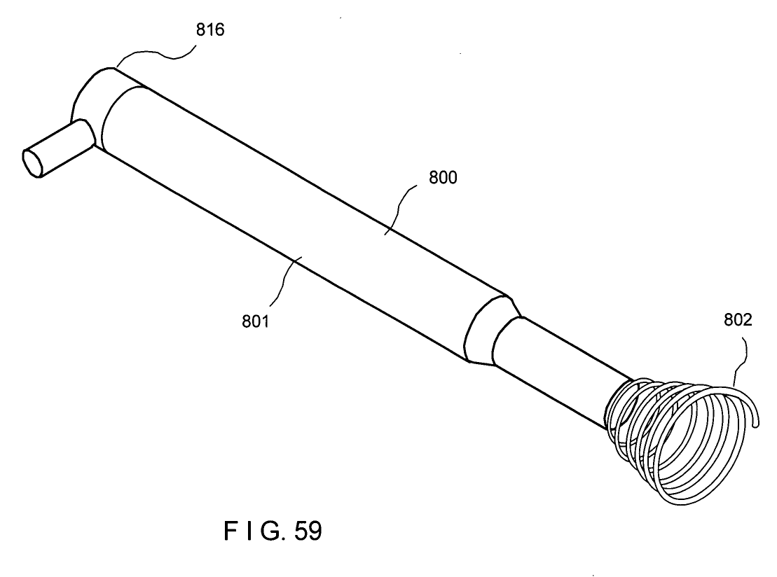

Yet another exemplary embodiment of the present invention is shown in FIG. 58. The exemplary cervical tenaculum tie 700b of FIG. 58 comprises a tether 716 adapted for connecting the two ratchets 702, 704a. The tether 716 limits the distance by which the two ratchets 702, 704a can be separated. This feature is useful because, if the two ratchets 702, 704a are separated by too great a distance, moving the movable ratchet 702 may cause the band 706 to buckle rather than moving through the stationary ratchet 704a. Limiting the distance between the ratchets 702, 704a decreases the likelihood of buckling. The tie 700b may preferentially retain a straightened shape prior to insertion of the end of the band 706 into the ratchets 702, 704a. This straightened configuration allows the entire tie 700b (or other similar ties) to be injection molded as a single piece. For example, a plastic material or other polymer may be used to form the tie.

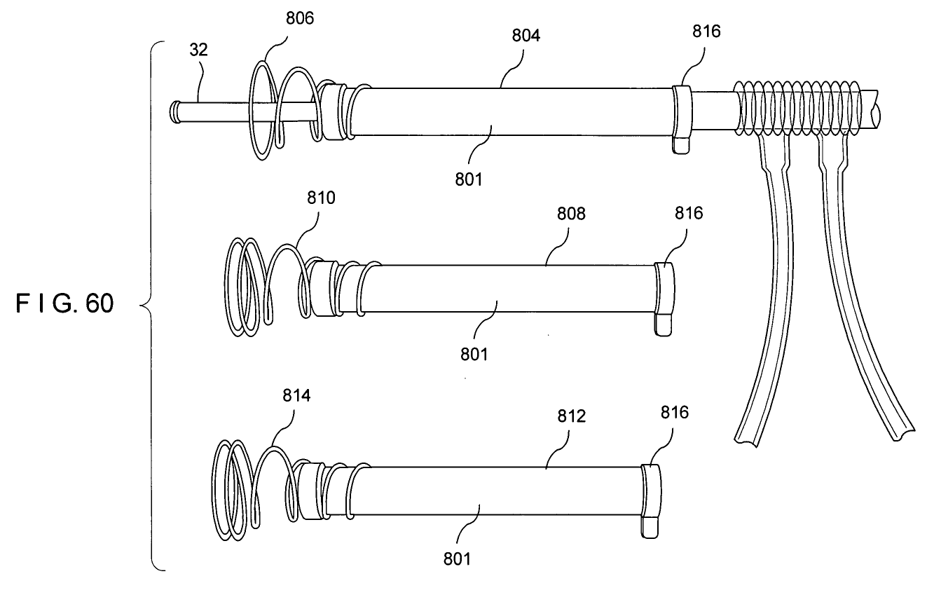

Additional embodiments of a cervical tenaculum according to the present invention are shown in FIGS. 59-61. These embodiments generally utilize a cinch-like device to provide the constriction force used to seal the cervical opening of a patient around a medical device inserted therethrough. As shown in FIG. 59, the cinch 800 comprises a hollow body 801 having a bore formed therethrough. A cap 816 is disposed at one end of the body 801, while the other end of the body 801 includes a coil 802 adapted to expand outwardly from the body 801. Either or both of the body 801 and the cap 816 may be made, for example, of a surgical metal such as stainless steel while the coil may be made, for example, from a metal wire.

During use of the exemplary tenaculum cinch 800 depicted in FIG. 60, a medical device 32 is inserted through the bore of the body 801 of the cinch 800 and inserted into the uterus via the cervix. The cinch 800 is then rotated about its axis so that the coil 802 engages the cervix catching cervical tissue in the pitch of the coil 802 and drawing this tissue to the narrower portion of the coil 802. This closes the cervix about the medical device 32 to create the desired seal between the device 32 and the cervix.

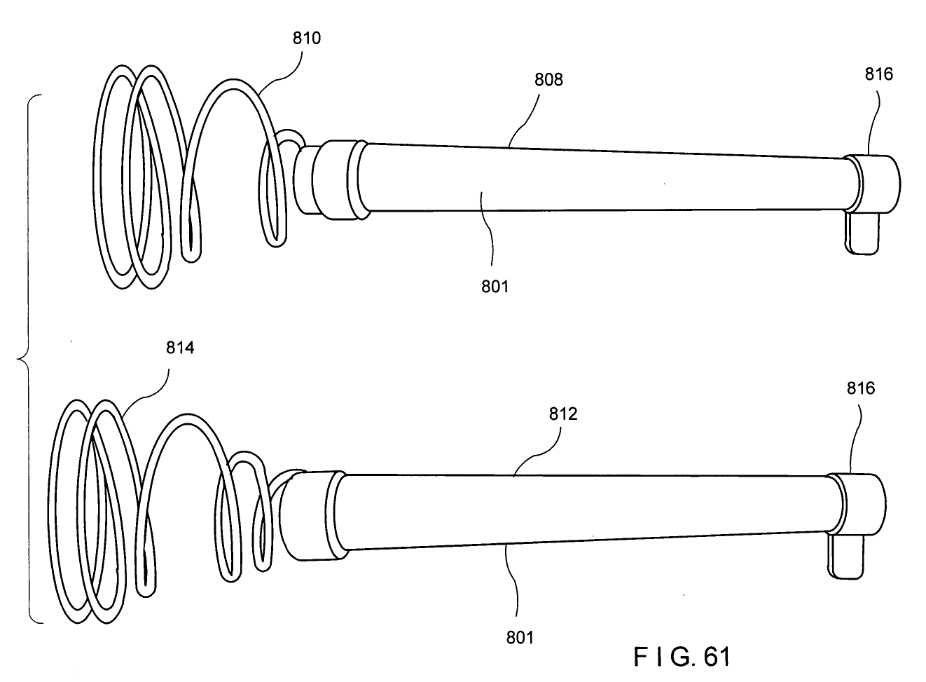

FIGS. 60 and 61 show additional variations of the exemplary tenaculum cinch, with cinches 804, 808 and 812 having different configurations of their respective coils 806, 810 and 812 which are located at the opposite ends of the caps 816 of each of these embodiments. Each of the coils 804, 808, 812 of the cinches 84, 808, 812, respectively, is affixed to the body 801 mechanically, for example by welding or using fasteners. More specifically, the coil 806 of the cinch 804 is shaped such that it revolves back onto itself after it makes about one outward revolution from the body 801. The coil 806 does not have a leading end as is shown in FIG. 59 (i.e., it is a closed coil). The coil 810 of cinch 808 is shaped such that it revolves back onto itself after it makes about two outward revolutions from the body 801. The coil 810 also does not have a leading end as is shown in FIG. 59 (i.e., it is a closed coil). The coil 814 of cinch 812 is shaped such that it revolves back onto itself after it makes about three outward revolutions from the body 801. As with the two prior embodiments, the coil 814 does not have a leading end as is shown in FIG. 59 (i.e., it is a closed coil). Despite these differences, the cinches 804, 808, 812 may be used by the surgeon in a manner similar to that described above with reference to the cinch 800.

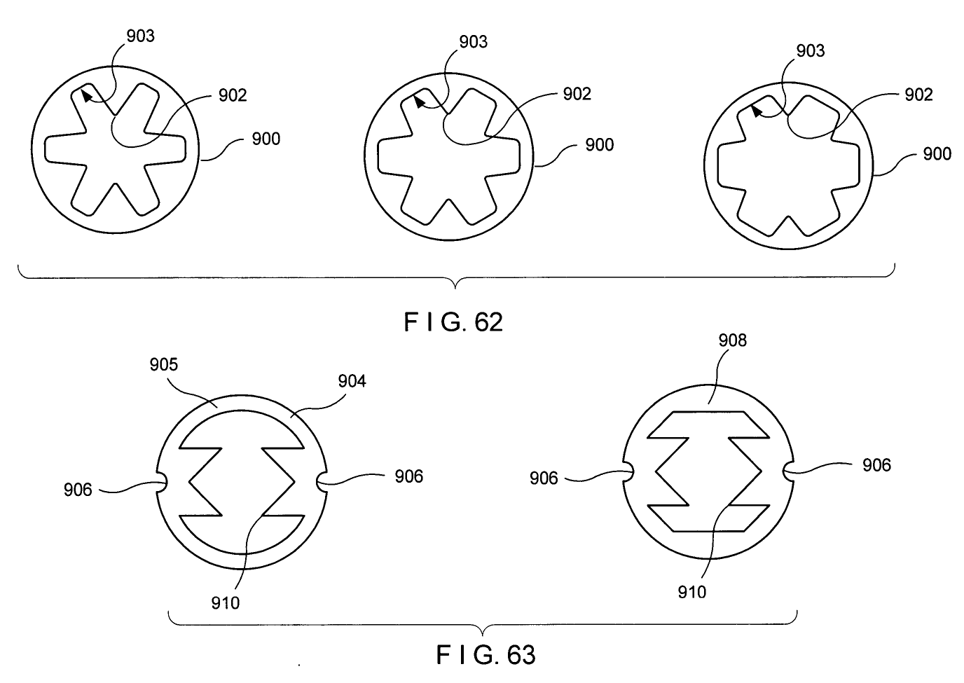

FIGS. 62 and 63 show exemplary embodiments of a cervical device that is generally tie-shaped. These embodiments may be manufactured in various sizes, as dictated by the range of sizes of a human cervix. An exemplary cervical tie 900 may be generally circular, and may comprise spikes 902 which extend from an inner side thereof. Between the spikes 902 hollows 903 are preferably formed. Depending on the size of the tie 900, the shape of the spikes 902 and the hollows 903 between the spikes 902 may vary, as seen in the slight difference of shapes in the embodiments of FIGS. 62 and 63. The tie 900 typically comprises a relatively thin planar surface, but may also be formed with additional depth so that it resembles a tube rather than a disk. As with the prior embodiments, the tie 900 may be made from any appropriate surgical material such as a plastic or metal, such as stamped steel. In use, the tie 900 is slid over the cervix after placing a medical device therethrough into the uterus. The tie 900 compresses the cervix about the medical device to form the seal around the medical device with the spikes 902 radially engaging the cervical tissue to prevent the tie 900 from slipping off of the cervix.

FIG. 63 shows a different embodiment of a cervical tenaculum that comprises a twist tab component. In one exemplary configuration, the twist tab 904 is generally circular and comprises prongs 910 that extend from the inner circumference of the rim 905. The twist tab 904 may comprise two indentations 906 where a medical clamp (such as a hemostat or forceps) may be placed to engage the rim 905. When the medical clamp is squeezed closed, the twist tab 904 deflects, and the prongs 910 swing away from the original plane of the twist tab 904. Releasing the squeezing pressure applied by the medical clamp allows the prongs 910 to return into the plane of the twist tab 904, thus applying a radially inward pressure to the portion of the cervix positioned therein.

In one exemplary procedure for using the cervical twist tab 904, the medical clamp is placed at the two indentations 906 and squeezed to open the twist tab 904. This action moves the prongs 910 away from the plane of the tab 904. While in this conformation, the twist tab 904 is placed over the cervix, through which a medical device has been inserted. The pressure applied by the medical clamp is then released to release the indentations 906 and allow the prongs 910 to return to their original substantially planar configuration. The return of the prongs 910 to their original shape clamps the cervix against the medical device, forming a seal that entirely surrounds the medical device. The tab may be formed in any of a variety of configurations to obtain a desired deflection of the prongs 910 as would be understood by those skilled in the art. In cases where an additional clamping force is desired around the cervix, the twist tab 904 may be deformed by applying a radially inward force at opposite locations approximately 90 degrees from the indentations 906. The exemplary tab 904 is preferably made of a stamped high tensile spring steel or other suitable materials. The twist tab 908 represents another exemplary configuration of prongs 910 and indentations, which functions in a fashion similar to that of the tab 904.

The present invention has been described with reference to specific exemplary embodiments. Those skilled in the art will understand that changes may be made in details, particularly in matters of shape, size, material and arrangement of parts. Accordingly, various modifications and changes may be made to the embodiments. For example, the exemplary devices described may be used to seal bodily cavities other than the cervix. The specifications and drawings are, therefore, to be regarded in an illustrative rather than a restrictive sense.

What is claimed is:

1. A cervical tenaculum comprising:
a base including a device receiving opening extending therethrough;
a plurality of arms, each arm extending from a proximal end connected to the base to a distal end adapted to apply radial pressure to a cervix; and
an arm closing element slidable along the arms between an open position in which the distal ends of the arms are released to a radially expanded configuration and a closed position in which the distal ends of the arms are radially constricted by the arm closing element with respect to the open position.

2. The cervical tenaculum according to claim 1, further comprising an arm lock element coupled to the arm closing element to immobilize the arm closing element in at least one of the open and closed positions.

3. The cervical tenaculum according to claim 1, further comprising a device lock operatively connected to the plurality of arms, the device lock selectively immobilizing a device extending through the device receiving opening relative to the base.

4. The cervical tenaculum according to claim 1, further comprising a first resilient element biasing the arm closing element toward the open position.

5. The cervical tenaculum according to claim 2, wherein the arm lock element comprises a second resilient element biasing the arm lock element toward the locking position.

6. The cervical tenaculum according to claim 3, wherein the device lock comprises first and second rotatable eccentric rings for frictionally engaging a device extending therethrough when the first ring is rotated relative to the second ring.

7. The cervical tenaculum according to claim 1, wherein the distal end of each arm further comprises an end effector for engaging and retaining cervical tissue.

8. The cervical tenaculum according to claim 7, wherein each of the end effectors comprises at least one tissue engaging spike.

9. The cervical tenaculum according to claim 1, wherein a closing force applied to the plurality of arms is adjustable by varying a position of the arm closing element along the arms.

10. The cervical tenaculum according to claim 1, wherein the arm closing element is a ring mounted around the plurality of arms so that, as the ring is moved further distally along the arms, the distal ends of the arms are drawn radially inward toward an inner circumference of the ring.

11. The cervical tenaculum according to claim 3, further comprising guide rods connecting the device lock to the arm closing element, the guide rods forming a frame defining a longitudinal device receiving passage therethrough.

12. The cervical tenaculum according to claim 2, further comprising a rapid release mechanism which, when actuated, rapidly returns the arm closing element from the closed position to the open position.

13. The cervical tenaculum according to claim 12, further comprising a manually operable lever coupled to the arm lock element for actuating the arm lock element to immobilize the arm closing element and a first resilient member biasing the arm closing element toward the open position.

14. The cervical tenaculum according to claim 1, wherein the base is formed of two separate halves joinable to form a unitary component.

15. A cervical sealing device, comprising:
an elongated frame with a distal end for placement adjacent to a cervix, the elongated frame defining a device receiving passage extending therethrough;
a constriction element coupled to the distal end of the elongated frame, the constriction element being operable between a constricted configuration for applying a radially inwardly directed force to the cervix and an open configuration in which the constriction element is loosened around the cervix; and
a manual control actuating the constriction element between the constricted and open configurations.

16. The cervical sealing device according to claim 15, further comprising a device lock mounted to the frame for movement between a first position extending into the device receiving passage for immobilizing a device received therein and an open position in which the device receiving passage is unobstructed.

17. The cervical sealing device according to claim 15, wherein the constriction element comprises a loop extending from the distal end, the loop being coupled to the manual control so that actuation of the manual control varies a diameter of the loop.

18. The cervical sealing device according to claim 17, further comprising control arms adapted to maintain the loop in a plane substantially perpendicular to an axis of the elongated frame.

19. The cervical sealing device according to claim 18, wherein the control arms bias the loop toward an open position.

20. The cervical sealing device according to claim 17, further comprising a pull wire connected to the loop, wherein operating the pull wire being tightens and loosens the loop.

21. The cervical sealing device according to claim 15, wherein the constriction element comprises a substantially planar clamp.

22. The cervical sealing device according to claim 21, wherein the substantially planar clamp includes attachment points for releasably connecting the clamp to control arms of the elongated frame.

23. The cervical sealing device according to claim 15, wherein the constriction element comprises movable arms having an open configuration and a closed configuration, the movable arms constricting the cervix in the closed configuration.

24. The cervical sealing device according to claim 23, further comprising an actuating block slidable along the movable arms, the actuating block controlling movement of the movable arms between the open and closed configurations.

25. The cervical sealing device according to claim 23, wherein the movable arms comprise resilient elements biasing the movable arms toward the open configuration.

26. The cervical sealing device according to claim 23, wherein the movable arms engage cervical tissue with a wire loop.

27. The cervical sealing device according to claim 23, wherein the movable arms engage cervical tissue with end effectors extending therefrom.

28. The cervical sealing device according to claim 18, further comprising protrusions extending from the control arms for engaging cervical tissue.

29. The cervical sealing device according to claim 23, further comprising protrusions extending from the movable arms for engaging cervical tissue.

30. A method of performing an intrauterine medical procedure, comprising:
placing a constricting element in proximity to a cervix;
introducing a device into a uterus through the cervix, the device extending through a passage of the constriction element;
placing a cervical constriction element of the constriction element in an operative position on the cervix; and
actuating the cervical constriction element to seal the cervix around the device.

31. The method according to claim 30, further comprising closing a plurality of arms of the cervical constriction element to apply a radially inwardly directed pressure around a periphery of the cervix.

32. The method according to claim 31, further comprising displacing an arm closing ring to simultaneously move each of the plurality of arms from an open position to a closed position.

33. The method according to claim 32, wherein the cervical constriction element includes a resilient element biasing the arms toward an open position.

34. The method according to claim 32, further comprising actuating an arm lock to prevent undesired movement of the arms.

35. The method according to claim 30, further comprising locking the device to the constriction element with a device lock.

36. The method according to claim 35, further comprising rotating an eccentric element of the constriction element lock to immobilize the device.

37. The method according to claim 34, further comprising releasing the arm lock to allow release and removal of the constriction element from the cervix.

38. The method according to claim 30, further comprising deploying a constriction loop of the constriction element around the cervix and around the device and tightening the constriction loop to apply a radial inward pressure to the cervix.

39. The method according to claim 38, further comprising controlling a shape and orientation of the constriction loop in an open position with a pair of control arms of the constriction element.

40. The method according to claim 39, wherein the constriction element includes a biasing member coupled to the pair of control arms biasing the constriction loop toward the open position.

41. The method according to claim 30, further comprising placing a constriction clamp in the operative position on the cervix.

42. The method according to claim 41, further comprising placing the constriction clamp over the cervix in an open position, and thereafter closing the constriction clamp.

43. The method according to claim 30, further comprising engaging cervical tissue with protrusions of the constriction element.
